SEQUENCE LISTING (113 sequences)

SEQ ID NO: 1 - protein, 4 aa, Artificial
  Synthetic Peptide
  MOD_RES (1)..(1): 4Hyp
  MOD_RES (4)..(4): AMIDATION
PLGW

SEQ ID NO: 2 - protein, 4 aa, Artificial
  Synthetic Peptide
  MOD_RES (1)..(1): 4Hyp
  MOD_RES (4)..(4): AMIDATION
PIGW

SEQ ID NO: 3 - protein, 4 aa, Artificial
  Synthetic Peptide
  MOD_RES (4)..(4): AMIDATION
PIGW

SEQ ID NO: 4 - protein, 4 aa, Artificial
  Synthetic Peptide
  MOD_RES (1)..(1): 3,4-dehydro-proline
  MOD_RES (4)..(4): AMIDATION
PIGW

SEQ ID NO: 5 - protein, 4 aa, Artificial
  Synthetic Peptide
  MOD_RES (4)..(4): AMIDATION
PLGW

SEQ ID NO: 6 - protein, 4 aa, Artificial
  Synthetic Peptide
  MOD_RES (4)..(4): AMIDATION
PLGY

SEQ ID NO: 7 - protein, 4 aa, Artificial
  Synthetic Peptide
  MOD_RES (4)..(4): AMIDATION
PRGW

SEQ ID NO: 8 - protein, 4 aa, Artificial
  Synthetic Peptide
  MOD_RES (4)..(4): AMIDATION
PWGW

SEQ ID NO: 9 - protein, 4 aa, Artificial
  Synthetic Peptide
  MOD_RES (4)..(4): AMIDATION
PIGY

SEQ ID NO: 10 - protein, 4 aa, Artificial
  Synthetic Peptide
  MOD_RES (4)..(4): AMIDATION
WPLG

SEQ ID NO: 11 - protein, 4 aa, Artificial
  Synthetic Peptide
  MOD_RES (4)..(4): AMIDATION
FPLG

SEQ ID NO: 12 - protein, 4 aa, Artificial
  Synthetic Peptide
  MOD_RES (1)..(1): 4-fluorophenylalanine
  MOD_RES (4)..(4): AMIDATION
FPLG

SEQ ID NO: 13 - protein, 4 aa, Artificial
  Synthetic Peptide
  MOD_RES (1)..(1): 4-chlorophenylalanine
  MOD_RES (4)..(4): AMIDATION
FPLG

SEQ ID NO: 14 - protein, 4 aa, Artificial
  Synthetic Peptide
  MOD_RES (1)..(1): 4-fluorophenylalanine
  MOD_RES (4)..(4): AMIDATION
FPIG

SEQ ID NO: 15 - protein, 4 aa, Artificial
  Synthetic Peptide
  MOD_RES (1)..(1): 4-fluorophenylalanine
  MOD_RES (2)..(2): 4Hyp
  MOD_RES (4)..(4): AMIDATION
FPLG

SEQ ID NO: 16 - protein, 4 aa, Artificial
  Synthetic Peptide
  MOD_RES (1)..(1): 4-fluorophenylalanine
  MOD_RES (2)..(2): 4Hyp
  MOD_RES (4)..(4): AMIDATION
FPIG

SEQ ID NO: 17 - protein, 4 aa, Artificial
  Synthetic Peptide
  MOD_RES (4)..(4): AMIDATION
WPLG

SEQ ID NO: 18 - protein, 4 aa, Artificial
  Synthetic Peptide
  MOD_RES (4)..(4): AMIDATION
WPIG

SEQ ID NO: 19 - protein, 4 aa, Artificial
  Synthetic Peptide
  MOD_RES (2)..(2): 4Hyp
  MOD_RES (4)..(4): AMIDATION
WPLG

SEQ ID NO: 20 - protein, 4 aa, Artificial
  Synthetic Peptide
  MOD_RES (2)..(2): 4Hyp
  MOD_RES (4)..(4): AMIDATION
WPIG

SEQ ID NO: 21 - protein, 5 aa, Artificial
  Synthetic Peptide
  MOD_RES (1)..(1): 4-fluorophenylalanine
  MOD_RES (5)..(5): AMIDATION
FYPLG

SEQ ID NO: 22 - protein, 5 aa, Artificial
  Synthetic Peptide
  MOD_RES (1)..(1): 4-chlorophenylalanine
  MOD_RES (5)..(5): AMIDATION
FYPLG

SEQ ID NO: 23 - protein, 5 aa, Artificial
  Synthetic Peptide
  MOD_RES (5)..(5): AMIDATION
FYPLG

SEQ ID NO: 24 - protein, 5 aa, Artificial
  Synthetic Peptide
  MOD_RES (5)..(5): AMIDATION
FYPIG

SEQ ID NO: 25 - protein, 5 aa, Artificial
  Synthetic Peptide
  MOD_RES (3)..(3): 4Hyp
  MOD_RES (5)..(5): AMIDATION
FYPLG

SEQ ID NO: 26 - protein, 5 aa, Artificial
  Synthetic Peptide
  MOD_RES (3)..(3): 4Hyp
  MOD_RES (5)..(5): AMIDATION
FYPIG

SEQ ID NO: 27 - protein, 5 aa, Artificial
  Synthetic Peptide
  MOD_RES (5)..(5): AMIDATION
YYPLG

SEQ ID NO: 28 - protein, 5 aa, Artificial
  Synthetic Peptide
  MOD_RES (5)..(5): AMIDATION
YYPIG

SEQ ID NO: 29 - protein, 5 aa, Artificial
  Synthetic Peptide
  MOD_RES (3)..(3): 4Hyp
  MOD_RES (5)..(5): AMIDATION
YYPLG

SEQ ID NO: 30 - protein, 5 aa, Artificial
  Synthetic Peptide
  MOD_RES (3)..(3): 4Hyp
  MOD_RES (5)..(5): AMIDATION
YYPIG

SEQ ID NO: 31 - protein, 5 aa, Artificial
  Synthetic Peptide
  MOD_RES (5)..(5): AMIDATION
FPLGW

SEQ ID NO: 32 - protein, 5 aa, Artificial
  Synthetic Peptide
  MOD_RES (5)..(5): AMIDATION
YPLGW

SEQ ID NO: 33 - protein, 5 aa, Artificial
  Synthetic Peptide
  MOD_RES (2)..(2): 4Hyp
  MOD_RES (5)..(5): AMIDATION
FPLGW

SEQ ID NO: 34 - protein, 5 aa, Artificial
  Synthetic Peptide
  MOD_RES (5)..(5): AMIDATION
FPIGW

SEQ ID NO: 35 - protein, 5 aa, Artificial
  Synthetic Peptide
  MOD_RES (2)..(2): 4Hyp
  MOD_RES (5)..(5): AMIDATION
FPIGW

SEQ ID NO: 36 - protein, 5 aa, Artificial
  Synthetic Peptide
  MOD_RES (2)..(2): 4Hyp
  MOD_RES (5)..(5): AMIDATION
YPLGW

SEQ ID NO: 37 - protein, 5 aa, Artificial
  Synthetic Peptide
  MOD_RES (5)..(5): AMIDATION
YPIGW

SEQ ID NO: 38 - protein, 5 aa, Artificial
  Synthetic Peptide
  MOD_RES (2)..(2): 4Hyp
  MOD_RES (5)..(5): AMIDATION
YPLGW

SEQ ID NO: 39 - protein, 5 aa, Artificial
  Synthetic Peptide
  MOD_RES (5)..(5): AMIDATION
YPWGW

SEQ ID NO: 40 - protein, 5 aa, Artificial
  Synthetic Peptide
  MOD_RES (2)..(2): 4Hyp
  MOD_RES (5)..(5): AMIDATION
YPWGW

SEQ ID NO: 41 - protein, 5 aa, Artificial
  Synthetic Peptide
  MOD_RES (1)..(1): 4-fluorophenylalanine
  MOD_RES (2)..(2): 4Hyp
  MOD_RES (5)..(5): AMIDATION
FPIGW

SEQ ID NO: 42 - protein, 5 aa, Artificial
  Synthetic Peptide
  MOD_RES (1)..(1): 4-fluorophenylalanine
  MOD_RES (2)..(2): 4Hyp
  MOD_RES (5)..(5): AMIDATION
FPLGW

SEQ ID NO: 43 - protein, 5 aa, Artificial
  Synthetic Peptide
  MOD_RES (1)..(1): 4-fluorophenylalanine
  MOD_RES (2)..(2): 4Hyp
  MOD_RES (5)..(5): AMIDATION
FPRGW

SEQ ID NO: 44 - protein, 5 aa, Artificial
  Synthetic Peptide
  MOD_RES (5)..(5): AMIDATION
PILGW

SEQ ID NO: 45 - protein, 5 aa, Artificial
  Synthetic Peptide
  MOD_RES (1)..(1): 4Hyp
  MOD_RES (5)..(5): AMIDATION
PILGW

SEQ ID NO: 46 - protein, 6 aa, Artificial
  Synthetic Peptide
  MOD_RES (1)..(1): 4-fluorophenylalanine
  MOD_RES (2)..(2): 4Hyp
  MOD_RES (6)..(6): AMIDATION
FPIGWG

SEQ ID NO: 47 - protein, 7 aa, Artificial
  Synthetic Peptide
  MOD_RES (1)..(1): 4-fluorophenylalanine
  MOD_RES (2)..(2): 4Hyp
  MOD_RES (7)..(7): AMIDATION
FPIGWGW

SEQ ID NO: 48 - protein, 6 aa, Artificial
  Synthetic Peptide
  MOD_RES (1)..(1): 4-fluorophenylalanine
  MOD_RES (2)..(2): 4Hyp
  MOD_RES (6)..(6): AMIDATION
FPLGWG

SEQ ID NO: 49 - protein, 7 aa, Artificial
  Synthetic Peptide
  MOD_RES (1)..(1): 4-fluorophenylalanine
  MOD_RES (2)..(2): 4Hyp
  MOD_RES (7)..(7): AMIDATION
FPLGWGW

SEQ ID NO: 50 - protein, 7 aa, Artificial
  Synthetic Peptide
  MOD_RES (7)..(7): AMIDATION
PIGWPIG

SEQ ID NO: 51 - protein, 6 aa, Artificial
  Synthetic Peptide
  MOD_RES (1)..(1): 4-fluorophenylalanine
  MOD_RES (2)..(2): 4Hyp
  MOD_RES (6)..(6): AMIDATION
FPRGWG

SEQ ID NO: 52 - protein, 7 aa, Artificial
  Synthetic Peptide
  MOD_RES (1)..(1): 4-fluorophenylalanine
  MOD_RES (2)..(2): 4Hyp
  MOD_RES (7)..(7): AMIDATION
FPRGWGW

SEQ ID NO: 53 - protein, 6 aa, Artificial
  Synthetic Peptide
  MOD_RES (1)..(1): 4Hyp
  MOD_RES (4)..(4): 4Hyp
  MOD_RES (6)..(6): AMIDATION
PIGPIG

SEQ ID NO: 54 - protein, 4 aa, Artificial
  Synthetic Peptide
  MOD_RES (4)..(4): AMIDATION
YPWG

SEQ ID NO: 55 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): AMIDATION
FPRGW

SEQ ID NO: 56 - protein, 7 aa, Artificial
  Synthetic Peptide
  MOD_RES (1)..(1): 4-fluorophenylalanine
  MOD_RES (2)..(2): 4Hyp
  MOD_RES (7)..(7): AMIDATION
FPRGIGW

SEQ ID NO: 57 - protein, 5 aa, Artificial
  Synthetic Peptide
  MOD_RES (1)..(1): 4-fluorophenylalanine
  MOD_RES (2)..(2): homo-proline
  MOD_RES (5)..(5): AMIDATION
FPRGW

SEQ ID NO: 58 - protein, 6 aa, Artificial
  Synthetic Peptide
  MOD_RES (1)..(1): 4-fluorophenylalanine
  MOD_RES (2)..(2): 4Hyp
  MOD_RES (6)..(6): AMIDATION
FPRGGW

SEQ ID NO: 59 - protein, 5 aa, Artificial
  Synthetic Peptide
  MOD_RES (1)..(1): 4-methoxyphenylalanine
  MOD_RES (2)..(2): 4Hyp
  MOD_RES (5)..(5): AMIDATION
FPRGW

SEQ ID NO: 60 - protein, 4 aa, Artificial
  Synthetic Peptide
  MOD_RES (1)..(1): 4-chlorophenylalanine
  MOD_RES (2)..(2): 4Hyp
  MOD_RES (4)..(4): AMIDATION
FPIG

SEQ ID NO: 61 - protein, 5 aa, Artificial
  Synthetic Peptide
  MOD_RES (1)..(1): 4-chlorophenylalanine
  MOD_RES (2)..(2): 4Hyp
  MOD_RES (5)..(5): AMIDATION
FPRGW

SEQ ID NO: 62 - protein, 4 aa, Artificial
  Synthetic Peptide
  MOD_RES (1)..(1): 3,4-dehydroproline
  MOD_RES (4)..(4): AMIDATION
PRGW

SEQ ID NO: 63 - protein, 5 aa, Artificial
  Synthetic Peptide
  MOD_RES (1)..(1): 4-aminophenylalanine
  MOD_RES (2)..(2): 4Hyp
  MOD_RES (5)..(5): AMIDATION
FPRGW

SEQ ID NO: 64 - protein, 5 aa, Artificial
  Synthetic Peptide
  MOD_RES (1)..(1): 4-fluorophenylalanine
  MOD_RES (2)..(2): 4Hyp
  MOD_RES (5)..(5): AMIDATION
FPHGW

SEQ ID NO: 65 - protein, 5 aa, Artificial
  Synthetic Peptide
  MOD_RES (1)..(1): 4-nitrophenylalanine
  MOD_RES (2)..(2): 4Hyp
  MOD_RES (5)..(5): AMIDATION
FPRGW

SEQ ID NO: 66 - protein, 5 aa, Artificial
  Synthetic Peptide
  MOD_RES (1)..(1): 3-fluorophenylalanine
  MOD_RES (2)..(2): 4Hyp
  MOD_RES (5)..(5): AMIDATION
FPRGW

SEQ ID NO: 67 - protein, 6 aa, Artificial
  Synthetic Peptide
  MOD_RES (1)..(1): 4-fluorophenylalanine
  MOD_RES (2)..(2): 4Hyp
  MOD_RES (6)..(6): AMIDATION
FPRIGW

SEQ ID NO: 68 - protein, 5 aa, Artificial
  Synthetic Peptide
  MOD_RES (1)..(1): 2-fluorophenylalanine
  MOD_RES (2)..(2): 4Hyp
  MOD_RES (5)..(5): AMIDATION
FPRGW

SEQ ID NO: 69 - protein, 5 aa, Artificial
  Synthetic Peptide
  MOD_RES (1)..(1): 4-fluorophenylalanine
  MOD_RES (2)..(2): homo-proline
  MOD_RES (5)..(5): AMIDATION
FPIGW

SEQ ID NO: 70 - protein, 5 aa, Artificial
  Synthetic Peptide
  MOD_RES (1)..(1): 3-(3-pyridyl)alanine
  MOD_RES (2)..(2): 4Hyp
  MOD_RES (5)..(5): AMIDATION
APRGW

SEQ ID NO: 71 - protein, 5 aa, Artificial
  Synthetic Peptide
  MOD_RES (1)..(1): 4-fluorophenylalanine
  MOD_RES (2)..(2): 4Hyp
  MOD_RES (3)..(3): homo-arginine
  MOD_RES (5)..(5): AMIDATION
FPRGW

SEQ ID NO: 72 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): AMIDATION
FPIGW

SEQ ID NO: 73 - protein, 5 aa, Artificial
  Synthetic Peptide
  MOD_RES (1)..(1): 4-fluorophenylalanine
  MOD_RES (2)..(2): 4Hyp
  MOD_RES (3)..(3): L-allo-isoleucine
  MOD_RES (5)..(5): AMIDATION
FPIGW

SEQ ID NO: 74 - protein, 5 aa, Artificial
  Synthetic Peptide
  MOD_RES (1)..(1): 4-fluorophenylalanine
  MOD_RES (2)..(2): 4Hyp
  MOD_RES (3)..(3): homo-arginine
FPRGW

SEQ ID NO: 75 - protein, 4 aa, Artificial
  Synthetic Peptide
  MOD_RES (1)..(1): 4-fluorophenylalanine
  MOD_RES (2)..(2): 4Hyp
  MOD_RES (4)..(4): glycine-1,2,3,4-tetrahydroisoquinoline-3-carboxamide
FPRG

SEQ ID NO: 76 - protein, 5 aa, Artificial
  Synthetic Peptide
  MOD_RES (1)..(1): 3,4-dichlorophenylalanine
  MOD_RES (2)..(2): 4Hyp
  MOD_RES (5)..(5): AMIDATION
FPRGW

SEQ ID NO: 77 - protein, 5 aa, Artificial
  Synthetic Peptide
  MOD_RES (1)..(1): 4-cyanophenylalanine
  MOD_RES (2)..(2): 4Hyp
  MOD_RES (5)..(5): AMIDATION
FPRGW

SEQ ID NO: 78 - protein, 5 aa, Artificial
  Synthetic Peptide
  MOD_RES (1)..(1): 4-fluorophenylalanine
  MOD_RES (2)..(2): 4Hyp
  MOD_RES (3)..(3): D-leucine
  MOD_RES (5)..(5): AMIDATION
FPLGW

SEQ ID NO: 79 - protein, 5 aa, Artificial
  Synthetic Peptide
  MOD_RES (1)..(1): 4-fluorophenylalanine
  MOD_RES (2)..(2): trans-3-hydroxyproline
  MOD_RES (5)..(5): AMIDATION
FPRGW

SEQ ID NO: 80 - protein, 6 aa, Artificial
  Synthetic Peptide
  MOD_RES (1)..(1): 4-fluorophenylalanine
  MOD_RES (2)..(2): 3,4-dehydroproline
  MOD_RES (6)..(6): AMIDATION
FPRGGW

SEQ ID NO: 81 - protein, 5 aa, Artificial
  Synthetic Peptide
  MOD_RES (1)..(1): 4-methoxyphenylalanine
  MOD_RES (2)..(2): 3,4-dehydroproline
  MOD_RES (5)..(5): AMIDATION
FPRGW

SEQ ID NO: 82 - protein, 5 aa, Artificial
  Synthetic Peptide
  MOD_RES (1)..(1): 2,4-difluorophenylalanine
  MOD_RES (2)..(2): 3,4-dihydroproline
  MOD_RES (5)..(5): AMIDATION
FPRGW

SEQ ID NO: 83 - protein, 5 aa, Artificial
  Synthetic Peptide
  MOD_RES (1)..(1): 4-trifluoromethylphenylalanine
  MOD_RES (2)..(2): 3,4-dehydroproline
  MOD_RES (5)..(5): AMIDATION
FPRGW

SEQ ID NO: 84 - protein, 5 aa, Artificial
  Synthetic Peptide
  MOD_RES (1)..(1): 4-fluorophenylglycine
  MOD_RES (2)..(2): 3,4-dehydroproline
  MOD_RES (5)..(5): AMIDATION
GPRGW

SEQ ID NO: 85 - protein, 5 aa, Artificial
  Synthetic Peptide
  MOD_RES (1)..(1): 3-flurotyrosine
  MOD_RES (2)..(2): 3,4-dehydroproline
  MOD_RES (5)..(5): AMIDATION
YPRGW

SEQ ID NO: 86 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): carboxyl-terminal hydroxyamino group
FPRGW

SEQ ID NO: 87 - protein, 5 aa, Artificial
  Synthetic Peptide
  MOD_RES (1)..(1): 3,4-dichlorophenylalanine
  MOD_RES (2)..(2): 3,4-dehydroproline
  MOD_RES (5)..(5): AMIDATION
FPRGW

SEQ ID NO: 88 - protein, 5 aa, Artificial
  Synthetic Peptide
  MOD_RES (1)..(1): 2-fluorotyrosine
  MOD_RES (2)..(2): 3,4-dehydroproline
  MOD_RES (5)..(5): AMIDATION
YPRGW

SEQ ID NO: 89 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 7-azatryptophan, carboxyl-terminal amidated
FPRGW

SEQ ID NO: 90 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 4-fluorotryptophan, carboxyl-terminal amidated
FPRGW

SEQ ID NO: 91 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 5-fluorotryptophan, carboxyl-terminal amidated
FPRGW

SEQ ID NO: 92 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 6-fluorotryptophan, carboxyl-terminal amidated
FPRGW

SEQ ID NO: 93 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 3-methoxytryptophan, carboxyl-terminal amidated
FPRGW

SEQ ID NO: 94 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): N-methyltryptophan, carboxyl-terminal amidated
FPRGW

SEQ ID NO: 95 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 1-methyltryptophan, carboxyl-terminal amidated
FPRGW

SEQ ID NO: 96 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 4-methyltryptophan, carboxyl-terminal amidated
FPRGW

SEQ ID NO: 97 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 5-methyltryptophan, carboxyl-terminal amidated
FPRGW

SEQ ID NO: 98 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 6-methyltryptophan, carboxyl-terminal amidated
FPRGW

SEQ ID NO: 99 - protein, 5 aa, Artificial
  Synthetic Peptide
  MOD_RES (1)..(1): 4-fluorophenylalanine
  MOD_RES (2)..(2): 3,4-dehydroproline
  MOD_RES (5)..(5): 5-hydroxytryptophan, carboxyl-terminal amidated
FPRGW

SEQ ID NO: 100 - protein, 5 aa, Artificial
  Synthetic Peptide
  MOD_RES (1)..(1): 4-fluorophenylalanine
  misc_feature (2)..(2): isonipecotic acid residue
  MOD_RES (5)..(5): AMIDATION
FXRGW

SEQ ID NO: 101 - protein, 5 aa, Artificial
  Synthetic Peptide
  MOD_RES (1)..(1): 4-fluorophenylalanine
  misc_feature (2)..(2): 2-carboxy-azetidine
  MOD_RES (5)..(5): AMIDATION
FXRGW

SEQ ID NO: 102 - protein, 5 aa, Artificial
  Synthetic Peptide
  MOD_RES (1)..(1): 4-fluorophenylalanine
  misc_feature (2)..(2): 1-amino-1-carboxycyclopentane
  MOD_RES (5)..(5): AMIDATION
FXRGW

SEQ ID NO: 103 - protein, 5 aa, Artificial
  Synthetic Peptide
  MOD_RES (1)..(1): 4-fluorophenylalanine
  misc_feature (2)..(2): 2-carboxy-aziridine
  MOD_RES (5)..(5): AMIDATION
FXRGW

SEQ ID NO: 104 - protein, 5 aa, Artificial
  Synthetic Peptide
  MOD_RES (1)..(1): 4-fluorophenylalanine
  misc_feature (2)..(2): 1-amino-1-carboxy-cyclopropyl residue
  MOD_RES (5)..(5): AMIDATION
FXRGW

SEQ ID NO: 105 - protein, 5 aa, Artificial
  Synthetic Peptide
  MOD_RES (1)..(1): 4-fluorophenylalanine
  misc_feature (2)..(2): 3-carboxy-1,4,5,6-tetrahydropyridine
  MOD_RES (5)..(5): AMIDATION
FXRGW

SEQ ID NO: 106 - protein, 5 aa, Artificial
  Synthetic Peptide
  MOD_RES (1)..(1): 4-fluorophenylalanine
  misc_feature (2)..(2): 2-carboxypyrrole
  MOD_RES (5)..(5): AMIDATION
FXRGW

SEQ ID NO: 107 - protein, 5 aa, Artificial
  Synthetic Peptide
  MOD_RES (1)..(1): 4-fluorophenylalanine
  MOD_RES (2)..(2): 4Hyp
  MOD_RES (5)..(5): 4-flurotryptophan, carboxyl-terminal amidated
FPRGW

SEQ ID NO: 108 - protein, 5 aa, Artificial
  Synthetic Peptide
  MOD_RES (1)..(1): 4-fluorophenylalanine
  MOD_RES (2)..(2): 4Hyp
  MOD_RES (5)..(5): 7-methyltryptophan, carboxyl-terminal amidated
FPRGW

SEQ ID NO: 109 - protein, 5 aa, Artificial
  Synthetic Peptide
  MOD_RES (1)..(1): 4-fluorophenylalanine
  MOD_RES (2)..(2): 4Hyp
  MOD_RES (3)..(3): 5,5,5-trifluoroleucine
  MOD_RES (5)..(5): AMIDATION
FPLGW

SEQ ID NO: 110 - protein, 5 aa, Artificial
  Synthetic Peptide
  MOD_RES (1)..(1): 4-fluorophenylalanine
  MOD_RES (2)..(2): 3,4-dehydroproline
  misc_feature (4)..(4): Sarcosine
  MOD_RES (5)..(5): AMIDATION
FPRXW

SEQ ID NO: 111 - protein, 3 aa, Artificial
  Synthetic Peptide
  MOD_RES (3)..(3): AMIDATION
PLG

SEQ ID NO: 112 - protein, 3 aa, Artificial
  Synthetic Peptide
PLG

SEQ ID NO: 113 - protein, 4 aa, Artificial
  Synthetic Peptide
  MOD_RES (4)..(4): AMIDATION
YPLG

(19) United States
(12) Patent Application Publication
(10) Pub. No.: US 2008/0004224 A1
(43) Pub. Date: Jan. 3, 2008

(54) DERMATOLOGICAL USES OF TRI-, TETRA-, PENTA-, AND POLYPEPTIDES

(76) Inventors: Joseph J. Hlavka, Tuxedo Park, NY (US); Martin F. Schacker, Huntington, NY (US)

Correspondence Address: DIAMOND LAW OFFICE LLC, 1605 JOHN STREET, SUITE 102, FORT LEE, NJ, 07024, US

(21) Appl. No.: 11/747,762

(22) Filed: May 11, 2007

Related U.S. Application Data
(63) Continuation-in-part of application No. 10/900,026, filed on Jul. 27, 2004, now abandoned.
Application No. 10/900,026 is a division of application No. 10/122,246, filed on Apr. 11, 2002, now Pat. No. 6,767,897.
Application No. 10/122,246 is a continuation-in-part of application No. 09/625,103, filed on Jul. 25, 2000, now abandoned.
Application No. 09/625,103 is a continuation-in-part of application No. 08/962,962, filed on Nov. 4, 1997, now Pat. No. 6,093,797.
Application No. 08/962,962 is a continuation-in-part of application No. 08/432,651, filed on May 2, 1995, now Pat. No. 5,767,083.
Application No. 08/432,651 is a continuation-in-part of application No. 08/238,089, filed on May 4, 1994, now Pat. No. 5,589,460.
(60) Provisional application No. 60/825,077, filed on Sep. 8, 2006.
(60) Provisional application No. 60/825,070, filed on Sep. 8, 2006.
(60) Provisional application No. 60/801,128, filed on May 17, 2006.

(51) Int. Cl. A61K 38/08; A61P 17/00

(52) U.S. Cl. 514/17

(57) ABSTRACT

The invention provides methods for improving the appearance and/or health of skin using topical compositions that include the tripeptide MIF (prolyl-leucyl-glycinamide) or modified peptide compounds based on MIF. Related aspects of the invention provide cosmetic and pharmaceutical topical compositions for the treatment of skin that contain one or more of the peptides.

This application claims the benefit of U.S. provisional patent application Ser. Nos. 60/825,077 and 60/825,070, each filed Sep. 8, 2006, and 60/801,128 filed May 17, 2006, and is a continuation-in-part of U.S. patent application Ser. No. 10/900,026 filed Jul. 27, 2004, which is a divisional of U.S. patent application Ser. No. 10/122,246 filed Apr. 11, 2002 (now U.S. Pat. No. 6,767,897), which is a continuation-in-part of U.S. patent application Ser. No. 09/625,103 filed Jul. 25, 2000 (abandoned), which is a continuation-in-part of U.S. patent application Ser. No. 08/962,962 filed Nov. 4, 1997 (now U.S. Pat. No. 6,093,797), which is a continuation-in-part of U.S. patent application Ser. No. 08/432,651 filed May 2, 1995 (now U.S. Pat. No. 5,767,083), which is a continuation-in-part of U.S. patent application Ser. No. 08/238,089 filed May 4, 1994 (now U.S. Pat. No. 5,589,460). Each of the foregoing patents and applications is incorporated by reference herein in its entirety.

### FIELD OF THE INVENTION

The invention relates generally to the field of dermatological treatments for improving the appearance and/or health of skin.

### BACKGROUND OF INVENTION

Melanocyte stimulating inhibitory factor, otherwise known as MIF, MIF-1 or PLG, is a naturally occurring tripeptide having the chemical formula prolyl-leucyl-glycinamide (L-prolyl-L-leucyl-glycinamide; Pro-Leu-Gly-NH₂). The tripeptide MIF is naturally present as the carboxyl terminal tripeptide of oxytocin. MIF has been shown to produce numerous non-endocrine effects on the brain and has been shown to be active in a number of animal models for depression.

MIF was initially isolated and characterized from bovine hypothalmic extracts (Nair, et al., 1971, Biochem. Biophys. Res. Comm. 43(6): 1376-1381) and rat hypothalmic extracts (Celis, et al., 1971, Proc. Natl. Acad. Sci. USA 68(7): 1428-1433). MIF activity was attributed to inhibiting release of melanocyte stimulating hormone, a pituitary hormone known to stimulate melanin production. Neither disclosure suggests or discloses any potential antidepressant activity for MIF.

U.S. Pat. No. 3,708,593 teaches that MIF exhibits antidepressant activity in mice, as shown by a modified Dopa test (Everett, et al., 1966, Proc. 1^{st} Int. Sym. Anti-Depressant Drugs, p. 164).

U.S. Pat. No. 3,795,738 teaches that MIF, alone or in combination with other known drugs, exhibits increased activity against Parkinson's disease.

U.S. Pat. No. 3,931,184 teaches isolation of medicinally pure MIF. A MIF hemihydrate is dissolved in methanol, followed by the addition of diethyl ether, resulting in a white crystalline precipitate of MIF. This pure MIF is collected, washed with ether and dried under vacuum prior to use.

U.S. Pat. No. 4,278,595 teaches that practical use of MIF has been hindered because MIF is rapidly metabolized subsequent to administration and discloses various MIF analogues.

Tyr-MIF-1 (Tyr-Pro-Leu-Gly-NH₂) is a brain derived peptide shown to affect passive avoidance in rats (Hayashi, et al., 1983, Brain Res. Bull. 11: 659-662). Various analogs to Tyr-MIF-1 (i.e., substitutions for the Tyr residue, resulting in Ala-MIF-1, Leu-MIF-1 or Phe-MIF-1) were tested for a possible effect on behavioral and motor activities (Hayashi, et al., 1984, Pharmacology Biochemistry & Behavior 21:809-812). Ala-MIF-1 and Phe-MIF-1, but not Leu-MIF-1, affected passive avoidance behavior in rats. None of these peptides were shown to affect motor behavior.

Kastin, et al. (1984, Pharmacology Biochemistry & Behavior 21: 767-771) discloses that MIF-1 and Tyr-MIF-1 are active as antidepressants. The degree of activity was measured by the water wheel test, a modification of the Porolt swim test.

Kastin, et al. (1985, Pharmacology Biochemistry & Behavior 23: 1045-1049) determined that Tyr-MIF-1 and several Tyr-MIF-1 analogs possess antiopiate activity. Along with Tyr-MIF-1, Phe-MIF-1 was active in inhibiting the analgesic effect of morphine in rats.

Banks, et al. (1986, Am. J. Physiol. 251 [Endocrine Metabolism 14]: E477-E482) identifies the carrier-mediated transport system responsible for delivery of Tyr-MIF-1 to the extracellular brain fluid from the circulatory system.

Petersson et al. (2001, Peptides. 22(9): 1479-84) discloses that oxytocin decreases carrageenan induced inflammation in rats.

Petersson et al. (2004, Physiol. Behav. 83(3): 475-81) discloses that prolyl-leucyl-glycinamide (MIF) shares some effects with oxytocin but decreases oxytocin levels in rats. However, this paper is silent with respect to topical application and effects of MIF.

Pahdy et al. (2005, Mediators Inflamm. (6): 360-5) discloses that oxytocin and melatonin inhibit Calotropis procera latex-induced inflammatory hyperalgesia in rats.

U.S. Pat. No. 6,358,929 discloses the use of a peptide for preventing intolerance reactions of the skin, in particular in cosmetic compositions.

U.S. Pat. No. 6,620,419 discloses cosmetic or dermo-pharmaceutical use of peptides for healing, hydrating and improving skin appearance during natural or induced aging.

U.S. Pat. No. 6,797,697 discloses compositions containing a peptide and a pigment, and the use thereof in darkening the skin.

U.S. Pat. No. 6,809,075 discloses elastin peptide analogs and their use in combination with skin enhancing agents.

U.S. Pat. No. 6,974,799 discloses pharmaceutical, personal care and cosmetic compositions for topical application that contain a tripeptide (Gly-His-Lys) and a tetrapeptide, and which are reportedly useful for treating visible signs of aging.

### SUMMARY OF INVENTION

One embodiment of the invention provides a topical composition that includes: a dermatologically acceptable vehicle; and one or more of prolyl-leucyl-glycinamide and the other peptides of this disclosure. The composition may be a pharmaceutical and/or cosmetic composition for the treatment of mammalian skin, such as human skin.

One embodiment of the invention provides a method for the cosmetic treatment of skin that includes the step of: applying a topical composition comprising one or more of prolyl-leucyl-glycinamide and the other peptides of this disclosure to a region of skin.

One embodiment of the invention provides a topical pharmaceutical composition that includes: a dermatologically acceptable vehicle; and a pharmaceutically-active amount of one or more of prolyl-leucyl-glycinamide and the other peptides of this disclosure.

One embodiment of the invention provides a method for treating an inflammatory skin condition that includes the step of: applying a topical composition comprising one or more of prolyl-leucyl-glycinamide and the other peptides of this disclosure to a region of skin in need of treatment for an inflammatory skin condition.

The compositions of the invention may contain, in addition to MIF and/or the other peptides of this disclosure, at least one of a moisturizing agent, a sunscreen agent, an anti-inflammatory agent, a cosmetic agent, a skin-enhancing or conditioning agent and any combination thereof.

One embodiment of the invention provides a method for preparing a topical composition for the treatment of skin, comprising the step of: admixing a dermatologically acceptable vehicle and an at least substantially pure preparation of one or more of prolyl-leucyl-glycinamide and the other peptides of this disclosure.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION

The invention provides topical compositions that include the tripeptide MIF (SEQ ID NO: 111) and/or one or more of the other peptides of this disclosure, methods of using the compositions and methods of preparing the compositions.

One embodiment of the invention provides a topical composition that includes: a dermatologically acceptable vehicle; and prolyl-leucyl-glycinamide and/or one or more of the other peptides of this disclosure. The composition may be a pharmaceutical and/or cosmetic composition for the treatment of mammalian skin, such as human skin.

One embodiment of the invention provides a method for the cosmetic treatment of skin that includes the step of: applying a topical composition comprising prolyl-leucyl-glycinamide and/or one or more of the other peptides of this disclosure to a region of skin. A related embodiment provides a method for the cosmetic treatment of skin that includes the step of: applying a topical composition comprising prolyl-leucyl-glycinamide and/or one or more of the other peptides of this disclosure to a region of skin, wherein said application reduces visible signs of aging of at least the region of skin to which the composition is applied. A variation of the embodiments provides for applying the composition on an at least once daily basis.

One embodiment of the invention provides a topical pharmaceutical composition that includes: a dermatologically acceptable vehicle; and a pharmaceutically-active amount of prolyl-leucyl-glycinamide and/or one or more of the other peptides of this disclosure.

One embodiment of the invention provides a method for treating an inflammatory skin condition that includes the step of: applying a topical composition comprising prolyl-leucyl-glycinamide and/or one or more of the other peptides of this disclosure to a region of skin in need of treatment for an inflammatory skin condition. A variation of the embodiment provides for applying the composition on an at least once daily basis.

While not being bound by theory, it is believed that topical application of MIF or the peptides of this disclosure will reduce sub-pathological and pathological inflammation of, or associated with, the skin, thereby improving the appearance and/or health of the treated skin.

Inflammatory conditions of the skin are prevalent. For example, the prevalence of psoriasis is 1-2% in the general population. Inflammatory conditions of the skin that may be treated with MIF according to the invention include, but are not limited to: psoriasis, eczema, atopic dermatitis, contact (allergic) dermatitis, keloid (hypertrophic scar), lichen simplex chronicus, prurigo nodularis, Reiter syndrome, pityriasis rubra pilaris, pityriasis rosea, stasis dermatitis, rosacea, acne, lichen planus, scleroderma, seborrheic dermatitis, granuloma annulare, dermatomyositis, alopecia areata, lichen planopilaris, vitiligo, and discoid lupus erythematosus.

MIF or the other peptides of this disclosure may be formulated in any type of vehicle (carrier) that is suitable for application and administration to the skin. Without limitation, MIF may, for example, be formulated into a cream (oil-in-water emulsion), lotion, ointment (water-in-oil emulsion), gel, foam, solution and/or suspension, such as those known in the art.

MIF or the other peptides of this disclosure may, for example, be formulated at an effective concentration within the pharmaceutical or cosmetic compositions of the invention in a range of about 0.0001% to about 90% by weight, or in a range of about 0.05% to about 50% by weight, or in a range of about 0.5% to about 10%, for example about 1.5% by weight. The compositions may also, for example, include MIF or the other peptides of this disclosure at a concentration in the range of 10⁻¹² M (molar) to 10⁻² M, or in a range from 10⁻⁷ M to 10⁻³ M.

### EXAMPLE 1

The following example illustrates a skin cream (oil-in-water emulsion) embodiment formulation of the invention that contains MIF.

| Component | Weight Amount | |
|---|---|---|
| Mixture (80:20) of cetylstearyl alcohol and | 5.0 | g |
| oxyethylenated cetylstearyl alcohol containing | | |
| 33 moles of oxyethylene | | |
| Glycerol monostearate | 1.5 | g |
| Cetyl alcohol | 0.75 | g |
| Liquid paraffin | 10.0 | g |
| Polydimethylsiloxane | 0.75 | g |
| Glycerin | 4.0 | g |
| Preservatives (optional) | qs | |
| MIF tripeptide | 5.0 | mg |
| Demineralized water | qs 100.0 | g |

Similar creams may, for example, be formulated with from 1 to 1,000 mg of MIF tripeptide.

### EXAMPLE 2

The following example illustrates a skin cream (oil-in-water emulsion) embodiment formulation of the invention that contains a pentapeptide having the formula 4-F-Phe-cis- or trans-4-OH-Pro-Arg-Gly-Trp-NH₂ [SEQ ID NO: 43].

| Component | Weight Amount | |
|---|---|---|
| Mixture (80:20) of cetylstearyl alcohol and | 5.0 | g |
| oxyethylenated cetylstearyl alcohol containing | | |
| 33 moles of oxyethylene | | |
| Glycerol monostearate | 1.5 | g |
| Cetyl alcohol | 0.75 | g |
| Liquid paraffin | 10.0 | g |
| Polydimethylsiloxane | 0.75 | g |
| Glycerin | 4.0 | g |
| Preservatives (optional) | qs | |
| 4-F-Phe-cis- or trans-4-OH-Pro-Arg-Gly-Trp-NH₂ | 5.0 | mg |
| Demineralized water | qs 100.0 | g |

Similar creams may, for example, be formulated with from 1 to 1,000 mg of the pentapeptide or similarly, for example, with one or more of any of the peptides according to the formulas of this disclosure.

MIF and the other peptides of this disclosure may also be combined with at least one dermatologically acceptable substance in a topical composition. The CTFA Cosmetic Ingredient Handbook, Ninth Edition (2002) describes a wide variety of non-limiting cosmetic and pharmaceutical ingredients commonly used in the skin care industry, which are suitable for use as additional ingredients in the compositions of the present invention. Additional ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantiveness of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents such as moisturizing agents, skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

One embodiment of the invention provides a composition for topical application that includes MIF and/or at least one of the other peptides of this disclosure, and at least one different dermatologically active substance, such as a moisturizing agent and/or an anti-inflammatory agent. Moisturizing agents may, for example, be selected from one or more of: occlusive agents; humectant agents; and emollients. Suitable occlusive moisturizing agents imlcude but are not limited to: fatty acids (e.g. lanolin acid); fatty alcohols (e.g. lanolin alcohol); hydrocarbon oils & waxes (e.g. petrolatum); polyhydric alcohols (e.g. propylene glycol); silicone (e.g. dimethicone); sterols (e.g. cholesterol); vegetable & animal fats (e.g. cocoa butter); vegetable and combinations thereof include, but are not limited to: aminobenzoic acid, padimate O, and oxybenzone combination; aminobenzoic acid and titanium dioxide combination; avobenzone, octocrylene, octyl salicylate, and oxybenzone combination; avobenzone and octyl methoxycinnamate combination; avobenzone, octyl methoxycinnamate, octyl salicylate, and oxybenzone combination; avobenzone, octyl methoxycinnamate, and oxybenzone combination; dioxybenzone, oxybenzone, and padimate O combination; homosalate; homosalate, menthyl anthranilate, and octyl methoxycinnamate combination; homosalate, menthyl anthranilate, octyl methoxycinnamate, octyl salicylate, and oxybenzone combination; homosalate, octocrylene, octyl methoxycinnamate, and oxybenzone combination; homosalate, octyl methoxycinnamate, octyl salicylate, and oxybenzone combination; homosalate, octyl methoxycinnamate, and oxybenzone combination; homosalate and oxybenzone combination; lisadimate, oxybenzone, and padimate O combination; lisadimate and padimate O combination; menthyl anthranilate; menthyl anthranilate, octocrylene, and octyl methoxycinnamate combination; menthyl anthranilate, octocrylene, octyl methoxycinnamate, and oxybenzone combination; menthyl anthranilate and octyl methoxycinnamate combination; menthyl anthranilate, octyl methoxycinnamate, and octyl salicylate combination; menthyl anthranilate, octyl methoxycinnamate, octyl salicylate, and oxybenzone combination menthyl anthranilate, octyl methoxycinnamate, and oxybenzone combination menthyl anthranilate and titanium dioxide combination; octocrylene and octyl methoxycinnamate combination; octocrylene, octyl methoxycinnamate, octyl salicylate, and oxybenzone combination; octocrylene, octyl methoxycinnamate, octyl salicylate, oxybenzone, and titanium dioxide combination; octocrylene, octyl methoxycinnamate, and oxybenzone combination; octocrylene, octyl methoxycinnamate, oxybenzone, and titanium dioxide combination; octocrylene, octyl methoxycinnamate, and titanium dioxide combination; octyl methoxycinnamate; octyl methoxycinnamate and octyl salicylate combination; octyl methoxycinnamate, octyl salicylate, and oxybenzone combination; octyl methoxycinnamate, octyl salicylate, oxybenzone, and padimate O combination; octyl methoxycinnamate, octyl salicylate, oxybenzone, padimate O, and titanium dioxide combination; octyl methoxycinnamate, octyl salicylate, oxybenzone, phenylbenzimidazole, and titanium dioxide combination; octyl methoxycinnamate, octyl salicylate, oxybenzone, and titanium dioxide combination; octyl methoxycinnamate, octyl salicylate, phenylbenzimidazole, and titanium dioxide combination; octyl methoxycinnamate, octyl salicylate, and titanium dioxide waxes (e.g. carnauba wax); and wax esters (e.g. bees wax). Suitable humectant moisturizing agents include but are not limited to: glycerol; honey; urea; alpha-propylene glycol; and alpha-hydroxy acids (and related combinations), such as glycolic acid, lactic acid, malic acid, citric acid, glycolic acid+ammonium glycolate, alpha-hydroxyethanoic acid+ammonium alpha-hydroxyethanoate, alpha-hydroxyoctanoic acid, alpha-hydroxycaprylic acid, hydroxycaprylic acid, mixed fruit acid, tri-alpha hydroxy fruit acids, triple fruit acid, sugar cane extract, L-alpha hydroxy acid. Squalene is an example of a suitable emollient.

Topical anti-inflammatory agents may, for example, include steroids (corticosteroids) such as, but not limited to: hydrocortisone (for example, 0.5-2.5% by weight); clobetasone (such as clobetasone butyrate); triamcinolone (such as triamcinolone acetonide); fluocinolone (such as fluocinolone acetonide); betamethasone valerate; betamethasone dipropionate; diflucortolone (such as diflucortolone valerate); fluticasone (such as fluticasone valerate); hydrocortisone 17-butyrate; mometasone (such as mometasone furoate); methylprednisolone (such as methylprednisolone aceponate); betamethasone dipropionate; and clobetasol (such as clobetasol propionate).

Non-steroidal anti-inflammatory agents useful in the topical composition of the invention include, but are not limited to: the oxicams, such as piroxicam, isoxicam, tenoxicam, sudoxicam, and CP-14, 304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

A further embodiment provides a topical composition that includes MIF and/or at least one of the other peptides of this disclosure, and a retinoid, such as tretinoin (all-trans retinoic acid).

Another embodiment of the invention provides a topical composition that includes MIF and a topical sunscreen agent or sunscreen agent combination. Suitable sunscreen agents combination; octyl methoxycinnamate and oxybenzone combination; octyl methoxycinnamate, oxybenzone, and padimate O combination; octyl methoxycinnamate, oxybenzone, padimate O, and titanium dioxide combination; octyl methoxycinnamate, oxybenzone, and titanium dioxide combination; octyl methoxycinnamate and padimate O combination; octyl methoxycinnamate and phenylbenzimidazole combination; octyl salicylate; octyl salicylate and padimate O combination; oxybenzone and padimate O combination; oxybenzone and roxadimate combination; padimate O; phenylbenzimidazole; phenylbenzimidazole and sulisobenzone combination; titanium dioxide; titanium dioxide and zinc oxide combination; and trolamine salicylate.

At least one other agent such as a moisturizing agent, a sunscreen agent, an anti-inflammatory agent, a cosmetic agent, a skin-enhancing agent and any combination thereof may also be mixed with the dermatologically acceptable vehicle and the at least substantially pure prolyl-leucyl-glycinamide and/or at least one of the other peptides of this disclosure, in the same mixing step or in a different mixing step.

MIF for use in the methods and compositions of the invention may be obtained in any manner. For example, MIF may be isolated or prepared from natural sources or may be prepared by peptide synthesis chemistry, such as by the methods known in the art. Preferably, MIF which is at least substantially pure is used in the preparation of the MIF-containing compositions of the invention. Accordingly, one embodiment of the invention provides a method for preparing a topical composition for the treatment of skin that includes the step of: mixing a dermatologically acceptable vehicle and an at least substantially pure quantity of prolyl-leucyl-glycinamide.

The peptides with which this invention is concerned are readily prepared by conventional procedures (i.e., carbodiimide method, mixed anhydride method, N, N-carbonyldiimidazole method) for the step-wise synthesis of polypeptides, and also including solid phase peptide synthesis. The substituent groups are also readily added to the polypeptide residues by conventional procedures. In general, peptides of this disclosure can be made using known amino acids, such as for example, Ala, Arg, D-Arg, Gly, Ile, Leu, D-Leu, Lys, Orn, Phe, Pro, dehydro-Pro, Sar, Trp, and Tyr. Preferred peptides are made by additions or substitutions at the amino terminus (N-terminus), carboxyl terminus (C-terminus) and/or additions or substitutions of internal amino acid residues with respect to the sequence Pro-Leu-Gly-NH₂ Carboxyl terminus modifications of the peptides of the invention can include replacement of the carbamyl (amide) group at the carboxyl terminus of Pro-Leu-Gly-NH₂ by, for example, a carboxyl (acid) group, a hydroxyalkyl (alcohol) group, an alkoxycarbonyl (ester) group, or an alkylcarbamyl (alkylated amide) group. Amino terminus and internal modifications of the peptides of the invention can include additions or eliminations on the heterocyclic, aromatic, and other carbon residues of the amino acids with an alkyl group, preferably an alkyl group having 1 to 3 carbon atoms, a dehydro (anhydro) group, a halo group, a hydroxyl group, a sulphydryl group, an alkylamino group, or a dialkylamino group. In some embodiments, the amino groups of the peptides of the invention can be alkylated, preferably with an alkyl group having 1 to 3 carbon atoms. Such additions, substitutions, eliminations, and/or modifications can, for example, be carried out by conventional polypeptide synthesis and organic chemistry synthesis techniques known to those skilled in the art.

Peptides and Peptide Formulas

Peptides including a carboxyl-terminal MIF sequence, Pro-Leu-Glycinamide, may also be used according to the invention instead of MIF itself or in addition to MIF. Thus, for any of the embodiments, variations and examples disclosed herein, peptides including the MIF sequence may be used instead of or in addition to MIF itself. Such peptides may, for example, be synthetic peptides.

In one embodiment, the peptide used is a peptide from three to nine amino acids, i.e., a tripeptide, tetrapeptide, peptapeptide, hexapeptide, heptapeptide, octapeptide or nonapeptide that includes the carboxyl terminal sequence Pro-Leu-Glycinamide. In one variation, the peptide is a peptide from four to nine amino acids in length. For example, the peptide may be Tyr-Pro-Leu-Glycinamide (SEQ ID NO: 113). In another variation, the peptide is a pentapeptide. The peptide may be a synthetic peptide. The term “carboxyl terminal sequence” as used herein means the sequence of a peptide of the invention occurring at the carboxy-terminus end of the peptide with respect to conventional peptide nomenclature irrespective of whether the particular peptide terminates with a carboxyl group.

Peptides that include the sequence Pro-Leu-Gly may also be used according to the invention instead of or in combination with MIF and/or any of the peptides disclosed herein. Thus, for any of the embodiments, variations and examples disclosed herein, peptides including the sequence Pro-Leu-Gly may be used instead of or in addition to MIF itself.

In one embodiment, the peptide is a peptide from three to nine amino acids in length that includes the sequence Pro-Leu-Gly. Thus, the tripeptide Pro-Leu-Gly (SEQ ID NO: 112) may be used. In one variation, the sequence Pro-Leu-Gly is present at the carboxyl terminus end of the peptide. In one variation, the sequence Pro-Leu-Gly is present at the amino terminus end of the peptide. In one variation, the peptide is a peptide from four to nine amino acids. In one variation, the peptide is a pentapeptide. The peptide may be a synthetic peptide.

In embodiments of the invention in which additional amino acids over a core peptide sequence, such as Pro-Leu-Glycinamide or Pro-Leu-Gly, are present, the additional amino acids may include without limitation the known naturally occurring amino acids (typically referred to by both their common three letter abbreviation and single letter abbreviation), stereoisomers, such as the D-forms, modifications of naturally occurring amino acids and -amide forms of amino acids.

Unless stated otherwise, the following terms and abbreviations when used herein have the meanings set forth below.

“Carboxyl” means any functional group having the formula —CO₂ H or —RCO₂ H, wherein R represents a monocyclic organic compound including a three to six member ring, of which at least one member is a nitrogen atom.

“Hydroxyalkyl” means any functional group having the formula —ROH, where R represents a lower alkyl group, preferably having 1 to 3 carbon atoms.

“Carbamyl” means any functional group having the formula —CONH₂ or —RCONH₂, wherein R represents a heterocyclic organic compound including a ten member ring, of which at least one member is a nitrogen atom.

“Alkylcarbamyl” means any functional group having the formula CONR¹R² wherein R¹ and R² each independently represent a hydrogen atom or a lower alkyl group, preferably having 1 to 3 carbon atoms.

“Alkoxycarbonyl” means any functional group having the formula CO₂ R, wherein R represents a lower alkyl group, preferably having 1 to 3 carbon atoms.

dehydro anhydro group where one or more hydrogen atoms are removed;

hydroxyl alcohol group or -OH or -ROH where R represents a lower alkyl group, preferably having 1 to 3 carbon atoms;

sulphydryl thiol group —SH or —RSH where R represents a lower alkyl group, preferably having 1 to 3 carbon atoms;

alkylamino NHR where R represents a lower alkyl group, preferably having 1 to 3 carbon atoms;

dialkylamino NR₂ where R represents a lower alkyl group, preferably having 1 to 3 carbon atoms;

hydroxyamino NHOH group;

patient includes any member of the animal kingdom, including but not limited to humans;

CGI Control group inactive;

Pro L-proline;

Leu L-leucine;

Gly L-glycine;

Tyr L-tyrosine;

Ala L-alanine;

Arg L-arginine;

Lys L-lysine;

Phe L-phenylalanine;

Trp L-tryptophan;

Ile L-isoleucine;

Om L-omithine;

D-Arg D-arginine;

D-Leu D-leucine;

3,4-dehydro-Pro 3,4-dehydro-L-proline;

pGlu pyro-glutamic acid;

Sar L-sarcosine (N-methylglycine);

4-OH-Pro 4-hydroxyproline;

4-thio-Pro 4-thioproline;

2-F-Phe 2-fluorophenylalanine;

3-F-Phe 3-fluorophenylalanine;

4-F-Phe 4-fluorophenylalanine;

4-Cl-Phe 4-chlorophenylalanine;

4-NH₂-Phe 4-aminophenylalanine;

3(3-pyridyl)Ala 3(3-pyridyl)-alanine;

Homo-Arg Homo-arginine;

Homo-Pro Homo-proline;

Fmoc 9-Fluorenylmethoxycarbonyl;

TFA trifluoroacetic acid.

One embodiment of the peptides of the present invention includes tripeptides of

formula (1):
R¹-Pro¹-AA¹-NR²—CH₂—R (1)
where Pro¹ represents the amino acid Pro or dehydro-Pro, preferably 3,4-dehydro Pro; AA¹ represents an amino acid of the group Trp, Orn, Lys, Leu, D-Leu, Arg, D-Arg, or Ile; R represents a carboxyl group, a hydroxyalkyl group, a carbamyl group, an alkylcarbamyl group, or an alkoxycarbonyl group; R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino group or dimethyl or diethylamino group; and, R² represents a hydrogen atom or a lower alkyl group, preferably having 1 to 3 carbon atoms, with the proviso that where Pro¹ is Pro and AA¹ is Leu, then R¹ and R² are not both hydrogen atoms when R is a carbamyl group.

An embodiment of peptides of formula (1) a tripeptide having formula (1a):
Pro¹-AA¹-Gly-NH₂ (1a)
wherein Pro¹ and AA¹ are as described above for formula (1). Examples of tripeptides according to formula (1a) include Pro-Trp-Gly-NH₂, Pro-Arg-Gly-NH₂, Pro-D-Arg-Gly-NH₂, Pro-Lys-Gly-NH₂, Pro-Orn-Gly-NH₂, and Pro-Ile-Gly-NH₂.

Another embodiment of tripeptides of formula (1) includes compounds of formula (1b):
R¹-Pro¹-AA¹-Gly-NH₂ (1b)
wherein Pro¹, AA¹ and R¹ are as described above for formula (1). Examples of tripeptides according to formula (1b) include cis- or trans-4-OH-Pro-D-Arg-Gly-NH₂, cis- or trans-4-OH-Pro-Ile-Gly-NH₂, cis- or trans-4-OH-Pro-Arg-Gly-NH₂, cis- or trans-4-OH-Pro-Trp-Gly-NH₂, and cis- or trans-4-thio-Pro-Leu-Gly-NH₂.

A further embodiment of tripeptides of formula (1) is formula (1c):
Pro¹-AA¹-NR¹—CH₂—R (1c)
wherein Pro¹, AA¹, R and R² are as described above for formula (1), with the proviso that where Pro¹ is Pro and AA¹ is Leu, R² is not a hydrogen atom when R is either a carboxyl group or a hydroxyalkyl group, and with the further proviso that where Pro¹ is Pro and AA¹ is Trp, R² is not a hydrogen atom when R is a hydroxyalkyl group. Examples of tripeptides according to formula (1c) include Pro-Leu-N(CH₃)CH₂—CONH₂ (or Pro-Leu-Sar-NH₂) and Pro-Trp-NHCH₂—CO₂H (or Pro-Trp-Gly).

In yet a further embodiment, are tripeptides represented by formula (2):
R¹-Pro¹-AA¹-Ala-R (2)
where Pro¹ represents the amino acid Pro or dehydro-Pro, preferably 3,4-dehydro Pro; AA¹ represents an amino acid of the group of Arg or D-Arg; R represents a carboxyl group, a hydroxyalkyl group, a carbamyl group, an alkylcarbamyl group, or an alkoxycarbonyl; and, R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

An embodiment of the tripeptides of formula (2) is represented by formula (2a):
Pro¹-AA¹-Ala-NH₂ (2a)
wherein Pro¹ and AA¹ are as described above for formula (2). Examples of tripeptides according to formula (2a) include Pro-Arg-Ala-NH₂ and Pro-D-Arg-Ala-NH₂.

In yet another embodiment, tripeptides of the present invention are represented by formula (3):
R¹-Pro¹-AA¹-Tyr-R (3)
where Pro¹ represents the amino acid Pro or dehydro-Pro, preferably 3,4-dehydro Pro; AA¹ represents the amino acid Orn; R represents a carboxyl group, a hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or a dimethyl or diethylamino group.

An embodiment of the tripeptides of formula (3) is formula (3a):
R¹-Pro¹-AA¹-Tyr-NH₂ (3a)
where Pro¹, AA¹ and R¹ are as described for formula (3). Examples of tripeptides according to formula (3a) include Pro-Orn-Tyr-NH₂ and cis- or trans-4-OH-Pro-Orn-Tyr-NH₂.

The present invention also provides tetrapeptides. One embodiment provides tetrapeptides represented by formula (4):
R¹-Pro¹-AA¹-Gly-AA²-R (4)
where Pro¹ represents the amino acid Pro or dehydro-Pro, preferably 3,4-dehydro Pro; AA¹ represents Ile, Leu, Arg, D-Arg or Trp; AA² represents an amino acid of the group of Trp or Tyr; R represents a carboxyl group, hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

An embodiment of tetrapeptides of formula (4) is formula (4a):
R¹-Pro¹-AA¹-Gly-AA²-NH₂ (4a)
wherein Pro¹, AA¹, AA², and R¹ are as described for formula (4). Examples of tetrapeptides according to formula (4a) include cis- or trans-4-OH-Pro-Leu-Gly-Trp-NH₂ (SEQ ID NO: 1), cis- or trans-4-OH-Pro-Ile-Gly-Trp-NH₂ (SEQ ID NO: 2), cis- or trans-4-OH-Pro-D-Arg-Gly-Trp-NH₂, 3,4-dehydro-Pro-D-Arg-Gly-Trp-NH₂ and 3,4-dehydro-Pro-Arg-Gly-Trp-NH2 (SEQ ID NO: 62).

A further embodiment of tetrapeptides of formula (4) is formula (4b):
Pro¹-AA¹-Gly-AA²-NH₂ (4b)
wherein Pro¹, AA¹ and AA² are as described for formula (4). Examples of tetrapeptides according to formula (4b) include Pro-Ile-Gly-Trp-NH₂ (SEQ ID NO: 3), 3,4-dehydro-Pro-Ile-Gly-Trp-NH₂ (SEQ ID NO: 4), Pro-Leu-Gly-Trp-NH₂ (SEQ ID NO: 5), Pro-Leu-Gly-Tyr-NH₂ (SEQ ID NO: 6), Pro-Arg-Gly-Trp-NH₂ (SEQ ID NO: 7), Pro-Trp-Gly-Trp-NH₂ (SEQ ID NO: 8), Pro-D-Arg-Gly-Trp-NH₂, and Pro-Ile-Gly-Tyr-NH₂ (SEQ ID NO: 9).

Another embodiment provides N-terminus end enhanced tetrapeptides represented by formula (5):
R¹-AA¹-R²-Pro¹-AA²-Gly-R (5)
where Pro¹ represents the amino acid Pro or dehydro-Pro, preferably 3,4-dehydro Pro; AA¹ represents an amino acid selected from Trp, Tyr and Phe; AA² represents an amino acid selected from Leu, Ile, and Trp; R represents a carboxyl group, hydroxyalkyl group, a carbamyl group, an alkylcarbamyl group, or an alkoxycarbonyl group; and, R¹ and R² each independently represent a hydrogen atom; a lower alkyl group, preferably having 1 to 3 carbon atoms; a halogen atom, preferably a fluorine or chlorine atom; a hydroxyl group; a sulphydryl group; or an alkylamino or dialkylamino group, preferably a methyl or ethylamino or dimethyl or diethylamino group.

One embodiment of tetrapeptides of formula (5) is represented by formula (5a):
R¹-AA¹-R²-Pro¹-AA²-Gly-NH₂ (5a)
wherein Pro¹, AA¹, AA², R¹ and R² are as described for formula (5), with the proviso that where Pro¹ is Pro, R¹ and R² are not both hydrogen atoms when AA¹ is Tyr and AA² is Trp; and with the further proviso that when Pro¹ is Pro, and AA² is Leu, and AA¹ is Phe or Tyr, then R¹ and R² are not both hydrogen atoms. Examples of tetrapeptides according to formula (5a) include Trp-Pro-Leu-Gly-NH₂ (SEQ ID NO: 10), Phe-Pro-Leu-Gly-NH₂ (SEQ ID NO: 11), 4-F-Phe-Pro-Leu-Gly-NH₂ (SEQ ID NO: 12), 4-Cl-Phe-Pro-Leu-Gly-NH₂ (SEQ ID NO: 13), 4-F-Phe-Pro-Ile-Gly-NH₂ (SEQ ID NO: 14), 4-F-Phe-cis- or trans-4-OH-Pro-Leu-Gly-NH₂ (SEQ ID NO: 15), 4-F-Phe-cis- or trans-4-OH-Pro-Ile-Gly-NH₂ (SEQ ID NO: 16), Trp-Pro-Leu-Gly-NH₂ (SEQ ID NO: 17), Trp-Pro-Ile-Gly-NH₂ (SEQ ID NO: 18), Trp-cis- or trans-4-OH-Pro-Leu-Gly-NH₂ (SEQ ID NO: 19), Trp-cis- or trans-4-OH-Pro-Ile-Gly-NH₂ (SEQ ID NO: 20), and 4-Cl-Phe-cis- or trans-4-OH-Pro-Ile-Gly-NH₂ (SEQ ID NO: 60).

Another embodiment of tetrapeptides of formula (5) is formula (5b):
R¹-AA¹-R²-Pro¹-AA²-Gly-R (5b)
wherein Pro¹, AA¹, AA², R¹, R² and R are as described for formula (5). Examples of tetrapeptides according to formula (5b) include compounds wherein the N-terminus heterocyclic nitrogen ring of Pro¹ is replaced by a cis- or trans-4-OH— group. In some embodiments of compositions represented by formula (5b), AA² is Arg. An example of a peptide of formula (5b) is 4-F-Phe-cis- or trans-4-OH-Pro-Arg-Gly-1,2,3,4-Tetrahydroisoquinoline-3-carboxamide (SEQ ID NO: 75).

The present invention further provides pentapeptides. One embodiment of the pentapeptides provides N-terminus enhanced pentapeptides represented by formula (6):
R¹-AA¹-AA²-R²-Pro¹-AA³-Gly-R (6)
where Pro¹ represents the amino acid Pro or dehydro-Pro, preferably 3,4-dehydro Pro; AA¹ and AA² each independently represent an amino acid of the group of Phe or Tyr; AA³ represents an amino acid of the group of Leu or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

An embodiment of the pentapeptides of formula (6) is formula (6a):
R¹-AA¹-AA²-R²-Pro¹-AA³-Gly-NH₂ (6a)
wherein Pro¹, AA¹, AA², R¹, and R² are as described for formula (6). Examples of pentapeptides of formula (6a) include 4-F-Phe-Tyr-Pro-Leu-Gly-NH₂ (SEQ ID NO: 21), 4-Cl-Phe-Tyr-Pro-Leu-Gly-NH₂ (SEQ ID NO: 22), Phe-Tyr-Pro-Leu-Gly-NH₂ (SEQ ID NO: 23), Phe-Tyr-Pro-Ile-Gly-NH₂ (SEQ ID NO: 24), Phe-Tyr-cis- or trans-4-OH-Pro-Leu-Gly-NH₂ (SEQ ID NO: 25); Phe-Tyr-cis- or trans-4-OH-Pro-Ile-Gly-NH₂ (SEQ ID NO: 26), Tyr-Tyr-Pro-Leu-Gly-NH₂ (SEQ ID NO: 27), Tyr-Tyr-Pro-Ile-Gly-NH₂ (SEQ ID NO: 28), Tyr-Tyr-cis- or trans-4-OH-Pro-Leu-Gly-NH₂ (SEQ ID NO: 29), and Tyr-Tyr-cis- or trans-4-OH-Pro-Ile-Gly-NH₂ (SEQ ID NO: 30).

Another embodiment of the pentapeptides provides combined N-terminus- and C-terminus-enhanced pentapeptides represented by formula (7):
R¹-AA¹-R²-Pro¹-AA²-Gly-AA³-R (7)
where Pro¹ represents the amino acid Pro or dehydro-Pro, preferably 3,4-dehydro Pro; AA¹ represents an amino acid of the group of Phe or Tyr; AA² represents an amino acid of the group of Leu, Ile, Arg, D-Arg, or Trp; AA³ represents the amino acid Trp; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

An embodiment of pentapeptides of formula (7) is represented by formula (7a):
R¹-AA¹-R²-Pro¹-AA²-Gly-Trp-NH₂ (7a)
wherein Pro¹, AA¹, AA², R¹ and R² are as described for formula (7). Examples of pentapeptides of formula (7a) include Phe-Pro-Leu-Gly-Trp-NH₂ (SEQ ID NO: 31), Tyr-Pro-Leu-Gly-Trp-NH₂ (SEQ ID NO: 32), Phe-cis- or trans-4-OH-Pro-Leu-Gly-Trp-NH₂ (SEQ ID NO: 33), Phe-Pro-Ile-Gly-Trp-NH₂ (SEQ ID NO: 34), Phe-cis- or trans-4-OH-Pro-Ile-Gly-Trp-NH₂ (SEQ ID NO: 35), Tyr-cis- or trans-4-OH-Pro-Leu-Gly-Trp-NH₂ (SEQ ID NO: 36), Tyr-Pro-Ile-Gly-Trp-NH₂ (SEQ ID NO: 37), Tyr-cis- or trans-4-OH-Pro-Leu-Gly-Trp-NH₂ (SEQ ID NO: 38), Tyr-Pro-Trp-Gly-Trp-NH₂ (SEQ ID NO: 39), Tyr-cis- or trans-4-OH-Pro-Trp-Gly-Trp-NH₂ (SEQ ID NO: 40), 4-F-Phe-cis- or trans-4-OH-Pro-Ile-Gly-Trp-NH₂ (SEQ ID NO: 41), 4-F-Phe-cis- or trans-4-OH-Pro-Leu-Gly-Trp-NH₂ (SEQ ID NO: 42), 4-F-Phe-cis- or trans-4-OH-Pro-Arg-Gly-Trp-NH₂ (SEQ ID NO: 43), 4-F-Phe-cis- or trans-4-OH-Pro-D-Arg-Gly-Trp-NH₂, 3-F-Phe-cis- or trans-4-OH-Pro-Arg-Gly-Trp-NH₂ (SEQ ID NO: 66); 2-F-Phe-cis- or trans-4-OH-Pro-Arg-Gly-Trp-NH₂ (SEQ ID NO: 68); and 4-Cl-Phe-cis- or trans-4-OH-Pro-Arg-Gly-Trp-NH₂ (SEQ ID NO: 61).

Additional examples of pentapeptides according to formula (7a) are characterized by the optional modification of Pro¹ to dehydro-Pro, preferably 3,4-dehydro-Pro. Additional preferred peptides of formula (7a) include 4-F-Phe-3,4-dehydro-Pro-Ile-Gly-Trp-NH₂ (SEQ ID NO: 72) and 4-F-Phe-3,4-dehydro-Pro-Arg-Gly-Trp-NH₂ (SEQ ID NO: 55).

Additional examples of pentapeptides of formula (7a) include pentapeptides having modifications at AA², preferably Arg, His, Homo-Arg, L-Allo-Ile or canavanine; additional optional modifications at R¹ and/or R² (preferably R¹) and preferably an amino group, a carboxyl group, a nitro group, or a phosphono group (preferably as phosphono-Try); additional optional modification of the heterocyclic nitrogen ring of Pro¹, preferably cis- or trans-4-OH or Homo-Pro. Exemplary additional preferred peptides of formula (7a) are 4-NH₂-Phe-cis- or trans-4-OH-Pro-Arg-Gly-Trp-NH₂ (SEQ ID NO: 63); 4-F-Phe-cis- or trans-4-OH-Pro-His-Gly-Trp-NH₂ (SEQ ID NO: 64); 4-NO₂-Phe-cis- or trans-4-OH-Pro-Arg-Gly-Trp-NH₂ (SEQ ID NO: 65); 4-CH₃ O-Phe-cis- or trans-4-OH-Pro-Arg-Gly-Trp-NH₂ (SEQ ID NO: 59); 4-F-Phe-cis- or trans-4-OH-Pro-Homo-Arg-Gly-Trp-NH₂ (SEQ ID NO: 71); 4-F-Phe-Homo-Pro-Ile-Gly-Trp-NH₂ (SEQ ID NO: 69); 4-F-Phe-Homo-Pro-Arg-Gly-Trp-NH₂ (SEQ ID NO: 57); and 4-F-Phe-cis- or trans-4-OH-Pro-L-Allo-Ile-Gly-Trp-NH₂ (SEQ ID NO: 73).

Another embodiment of pentapeptides of formula (7) is formula (7b):
R¹-AA¹-R²-Pro¹-AA²-Gly-Trp-NH₂ (7b)
wherein AA¹ is Phe; and Pro¹, AA², R¹ and R² are as described for formula (7), with the optional modification of the N-terminus heterocyclic nitrogen ring of Pro¹ with a substituent selected from the group consisting of cis-4-OH—, trans-4-OH—, cis-3-OH— and trans-3-OH. In some embodiments, the pentapeptides may be further modified at R¹, preferably by two or more halogen atoms, or a cyano group. Examples of pentapeptides of formula (7b) include 3,4-Dichloro-Phe-cis- or trans-4-OH-Pro-Arg-Gly-Trp-NH₂ (SEQ ID NO: 76), 4-NC-Phe-cis- or trans-4-OH-Pro-Arg-Gly-Trp-NH₂ (SEQ ID NO: 77), 4-F-Phe-cis- or trans-4-OH-Pro-D-Leu-Gly-Trp-NH₂ (SEQ ID NO: 78), 4-F-Phe-trans-3-Hydroxy-Pro-Arg-Gly-Trp-NH₂ (SEQ ID NO: 79).

Another embodiment of pentapeptides of formula (7) is formula (7c):
R¹-AA¹-R²-Pro¹-AA²-Gly-Trp (7c)
wherein Pro¹, AA¹, AA², R¹ and R² are as described for formula (7) with additional optional modifications at AA², preferably Homo-Arg; and additional optional modification of the N-terminus heterocyclic nitrogen ring of Pro¹ with a substituent selected from the group consisting of cis-4-OH—, trans-4-OH—, cis-4-OH— and trans-3-OH—. An example of a pentapeptide of formula (7c) is 4-F-Phe-cis- or trans-4-OH-Pro-Homo-Arg-Gly-Trp (SEQ ID NO: 74).

Another embodiment of pentapeptides of formula (7) is formula (7d):
R¹-AA¹-R²-Pro¹-AA²-Gly-AA³-R (7d)
wherein Pro¹, AA¹, AA², AA³, R¹, R² and R are as described for formula (7). In some embodiments, the N-terminus heterocyclic nitrogen ring of Pro¹ has a substituent selected from the group consisting of cis-4-OH—, trans-4-OH—, cis-3-OH—, and trans-3-OH—. In some embodiments the pentapeptide is modified at, Pro¹, preferably Homo-Pro. In some embodiments, the pentapeptide is modified at AA¹, preferably PhenylGly. In some embodiments, the pentapeptide is modified at AA³. In some embodiments, the pentapeptide is modified at R¹, preferably a haloform or a methoxyl group. In some embodiments, the pentapeptide is modified at R, preferably two or more halogen atoms or a hydroxyamino group.

Examples of pentapeptides of formula (7d) include 4-CH₃ O-Phe-3,4-Dehydro-Pro-Arg-Gly-Trp-NH₂ (SEQ ID NO: 81), 2,4-Di-F-Phe-3,4-Dihydro-Pro-Arg-Gly-Trp-NH₂ (SEQ ID NO: 82), 4-CF₃-Phe-3,4-Dehydro-Pro-Arg-Gly-Trp-NH₂ (SEQ ID NO: 83), 4-F-PhenylGly-3,4-Dehydro-Pro-Arg-Gly-Trp-NH₂ (SEQ ID NO: 84), 3-F-Tyr-3,4-Dehydro-Pro-Arg-Gly-Trp-NH₂ (SEQ ID NO: 85), 4-F-Phe-3,4-Dehydro-Pro-Arg-Gly-Trp-NHOH (SEQ ID NO: 86), 3,4-Di-Cl-Phe-3,4-Dihydro-Pro-Arg-Gly-Trp-NH₂ (SEQ ID NO: 87), 2-F-Tyr-3,4-Dehydro-Pro-Arg-Gly-Trp-NH₂ (SEQ ID NO: 88), 4-F-Phe-3,4-Dehydro-Pro-Arg-Gly-7-AzaTrp-NH₂ (SEQ ID NO: 89).

Another embodiment of pentapeptides of formula (7) is formula (7e):
R¹-AA¹-R²-Pro¹-AA²-Gly-R⁴-Trp-NH₂ (7e)
wherein Pro¹, AA¹, AA², R¹ and R² are as described for formula (7). Optionally, the N-terminus heterocyclic nitrogen ring of Pro¹ may be modified with a substituent selected from the group consisting of cis-4-OH—, trans-4-OH—, cis-3-OH—, and trans-3-OH—. R⁴ represents a modification of the tryptophan residue at one of C4, C5, C6 and C7 with a halogen atom, a hydroxyl group, or an alkyl group. Examples of pentapeptides of formula (7e) include 4-F-Phe-cis- or trans-4-OH-Pro-Arg-Gly-4-F-Trp-NH₂ (SEQ ID NO: 107), 4-F-Phe-cis- or trans-4-OH-Pro-Arg-Gly-7-Methyl-Trp-NH₂ (SEQ ID NO: 108).

Another embodiment of pentapeptides of formula (7) is formula (7f):
R¹-AA¹-R²-Pro¹-R⁵-AA²-Gly-Trp-NH₂ (7f)
wherein Pro¹, AA¹, AA², R¹ and R² are as described for formula (7). In some embodiments, the N-terminus heterocyclic nitrogen ring of Pro¹ is modified with a substituent selected from the group consisting of cis-4-OH—, trans-4-OH—, cis-3-OH—, and trans-3-OH—. R⁵ represents at least one halogen atom. An example of a pentapeptide of formula (7f) is 4-F-Phe-cis- or trans-4-OH-Pro-5,5,5-Trifluoro-Leu-Gly-Trp-NH₂ (SEQ ID NO: 109).

Another embodiment of pentapeptides of formula (7) is formula (7g):
R¹-AA¹-R²-Pro¹-AA²-Gly-R⁴-AA³-R (7g)
wherein Pro¹, AA¹, AA², AA³, R¹, R² and R are as described for formula (7). In some embodiments, Pro¹ is modified preferably Homo-Pro. In some embodiments, the N-terminus heterocyclic nitrogen ring of Pro¹ is modified with a substituent selected from the group consisting of cis-4-OH—, trans-4-OH—, cis-3-OH—, and trans-3-OH—. R⁴ represents a halogen atom, a methyl group, a methoxyl group or a hydroxyl group. Examples of pentapeptides of formula (7g) include 4-F-Phe-3,4-Dehydro-Pro-Arg-Gly-4-F-Trp-NH₂ (SEQ ID NO: 90), 4-F-Phe-3,4-Dehydro-Pro-Arg-Gly-5-F-Trp-NH₂ (SEQ ID NO: 91), 4-F-Phe-3,4-Dihydro-Pro-Arg-Gly-6-F-Trp-NH₂ (SEQ ID NO: 92), 4-F-Phe-3,4-Dehydro-Pro-Arg-Gly-3-CH₃ O-Trp-NH₂ (SEQ ID NO: 93), 4-F-Phe-3,4-Dehydro-Pro-Arg-Gly-N-Methyl-Trp-NH₂ (SEQ ID NO: 94), 4-F-Phe-3,4-Dehydro-Pro-Arg-Gly-1-Methyl-Trp-NH₂ (SEQ ID NO: 95), 4-F-Phe-3,4-Dehydro-Pro-Arg-Gly-4-Methyl-Trp-NH₂ (SEQ ID NO: 96), 4-F-Phe-3,4-Dehydro-Pro-Arg-Gly-5-Methyl-Trp-NH₂ (SEQ ID NO: 97), 4-F-Phe-3,4-Dehydro-Pro-Arg-Gly-6-Methyl-Trp-NH₂ (SEQ ID NO: 98), 4-F-Phe-3,4-Dehydro-Pro-Arg-Gly-5-Hydroxy-Trp-NH₂ (SEQ ID NO: 99).

Another embodiment provides internal and C-terminus enhanced pentapeptides represented by formula (8):
R¹-Pro¹-AA¹-AA²-Gly-AA³-R (8)
where Pro¹ represents the amino acid Pro or dehydro-Pro, preferably 3,4-dehydro Pro; AA¹ and AA² each independently represent an amino acid of the group of Leu or Ile; AA³ represents the amino acid Trp; R represents a carboxyl group, a hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino group or dimethyl or diethylamino group.

An embodiment of pentapeptides of formula (8) is formula (8a):
R¹-Pro¹-AA¹-AA²-Gly-Trp-NH₂ (8a)
wherein Pro¹, AA¹, AA², and R¹ are as described for formula (8). Examples of pentapeptides of formula (8a) include Pro-Ile-Leu-Gly-Trp-NH₂ (SEQ ID NO: 44) and cis- or trans-4-OH-Pro-Ile-Leu-Gly-Trp-NH₂ (SEQ ID NO: 45).

In another embodiment of the invention, pentapeptides or pharmaceutically acceptable salts thereof including addition of both a N-terminus amino acid and a C-terminus amino acid can be represented by the following formula (9):
R¹-AA¹-R²-Pro¹-AA²-Gly-AA³-R (9)
where Pro¹ represents the amino acid Pro or dehydro-Pro, preferably 3,4-dehydro Pro; AA¹ represents the amino acid Ala; AA² represents an amino acid of the group of Leu, Ile, Arg, D-Arg, Trp, or canavanine; AA³ represents the amino acid Trp; R represents a carboxyl group, hydroxyalkyl group, a carbamyl group, an alkylcarbamyl group, or an alkoxycarbonyl group; and, R¹ represents a pyridyl ring, preferably as a 3-(3-pyridyl) moiety; R² represents a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

An example of a peptide of formula (9) is 3-(3-pyridyl)-Ala-cis- or trans-4-OH-Pro-Arg-Gly-Trp-NH₂ (SEQ ID NO: 70).

In another embodiment of the invention, hexapeptides or pharmaceutically acceptable salts thereof including addition of both a N-terminus amino acid and a C-terminus amino acid can be represented by the following formula (10):
R¹-AA¹-R²-Pro¹-AA²-AA⁴-Gly-AA¹-R (10)
where Pro¹ represents the amino acid Pro or dehydro-Pro; AA¹ represents an amino acid of the group of Phe or Tyr; AA² represents an amino acid of the group of Leu, Ile, Arg, D-Arg, Trp, or canavanine; AA³ represents the amino acid Trp; AA⁴ represents the amino acid Gly or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

An example of a peptide of formula (10) is 4-F-Phe-3,4-Dehydro-Pro-Arg-Gly-Gly-Trp-NH₂ (SEQ ID NO: 80).

A group of hexapeptides of formula (10) includes hexapeptides characterized by addition of a C-terminus amino acid of Trp, optional modification of the heterocyclic nitrogen ring of Pro¹, preferably a cis- or trans-4-OH group, a fluorine atom at position 4 of Phe. Preferably, the hexapeptides include Arg at AA²; Trp at AA³; and Ile or Gly at AA⁴, and the C-terminus amide remains unmodified. Exemplary hexapeptides are represented by formula (10a):
R¹-Phe-R²-Pro¹-AA²-AA⁴-Gly-Trp-NH₂ (10a)
wherein R¹, R², AA², and AA⁴ are as defined above for formula (1). Examples of peptides of formula (10a) include 4-F-Phe-cis- or trans-4-OH-Pro-Arg-Gly-Gly-Trp-NH₂ (SEQ ID NO: 58) and 4-F-Phe-cis- or trans-4-OH-Pro-Arg-Ile-Gly-Trp-NH₂ (SEQ ID NO: 67).

Another embodiment of the invention provides heptapeptides or pharmaceutically acceptable salts thereof, including addition of both a N-terminus amino acid and a C-terminus amino acid can be represented by the following formula (11):
R¹-AA¹-R²-Pro¹-AA²-AA⁴-AA⁵-Gly-AA³-R (11)
where Pro¹ represents the amino acid Pro or dehydro-Pro; AA¹ represents an amino acid of the group of Phe or Tyr; AA² represents an amino acid of the group of Leu, Ile, Arg, D-Arg, Trp, or canavanine; AA³ represents the amino acid Trp; AA⁴ and AA⁵ represent the amino acid Gly or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group, or a phosphono group (preferably as phosphono-tyrosine).

An example of a peptide of formula (11) is 4-F-Phe-cis- or trans-4-OH-Pro-Arg-Gly-Ile-Gly-Trp-NH₂ (SEQ ID NO: 56).

In another embodiment of the invention, tetrapeptides or pharmaceutically acceptable salts thereof including the addition of an N-terminus amino acid of Phe to Arg; addition of a C-terminus amino acid of Trp to Gly; and modification of the aromatic ring of Phe can be represented by the following formula (12):
R¹-Phe-R²-Arg-Gly-Trp-NH₂ (12)
where R¹ represents a halogen atom and R² represents a carboxylic acid of a monocyclic organic compound with a three to six membered ring structure having a hetero nitrogen atom. Examples of pentapeptides of formula (12) include, but are not necessarily limited to, 4-F-Phe-isonipecotic acid-Arg-Gly-Trp-NH₂ (4-pyridinecarboxylic acid) (SEQ ID NO: 100), 4-F-Phe-2-Carboxy-Azetidine-Arg-Gly-Trp-NH₂ (SEQ ID NO: 101), 4-F-Phe-2-carboxy-Aziridine-Arg-Gly-Trp-NH₂ (SEQ ID NO: 103), 4-F-Phe-3-Carboxy-1,4,5,6-Tetrahydropyridine-Arg-Gly-Trp-NH₂ (SEQ ID NO: 105), 4-F-Phe-2-Carboxypyrrole-Arg-Gly-Trp-NH₂ (SEQ ID NO: 106).

In another embodiment of the invention, pentapeptides or pharmaceutically acceptable salts thereof including replacement of Pro with Arg; addition of an N-terminus amino acid of Phe to Arg; addition of an internal amino acid; addition of a C-terminus amino acid of Trp to Gly; and modification of the aromatic ring of Phe can be represented by the following formula (13):
R¹-Phe-AA¹-Arg-Gly-Trp-NH₂ (13)
where AA¹ represents an amino acid selected from the group comprising 1-amino-1-carboxycyclopentane and 1-amino-1-carboxy-cyclopropyl and R¹ represents a halogen atom. Examples of pentapeptides of formula (13) include 4-F-Phe-1-Amino-1-Carboxycyclopentane-Arg-Gly-Trp-NH₂ (SEQ ID NO: 102) and 4-F-Phe-1-Amino-1-Carboxy-Cyclopropyl-Arg-Gly-Trp-NH₂ (SEQ ID NO: 104).

In embodiments of peptides represented by any of formula (7), (8), (9), (10), or (11), Gly may be replaced by Val, Sar or Ala. One peptide wherein Gly is substituted with Sar in formula (7) is 4-F-Phe-3,4-Dehydro-Pro-Arg-Sar-Trp-NH₂ (SEQ ID NO: 110).

Further tetrapeptides that may be used in accordance with the invention are represented by the formula:
R¹-Phe-Pro¹-AA²-AA¹-NH₂
wherein R¹ is preferably a halogen atom, most preferably a fluorine or chlorine atom, a carboxyl group, an amino group or a nitro group, with all modifications preferably at the C4 atom of Phe; Pro¹ is 3,4-dehydro Pro, Homo-Pro, cis- or trans-4OH-Pro or Pro, as listed in order of preference, AA² is preferably Ile, Leu or Arg; and AA³ is preferably Gly or Trp.

A highly preferred tetrapeptide is Pro-Ile-Gly-Trp (SEQ ID NO: 3).

Further pentapeptides, hexapeptides and heptapeptides that may be used in accordance with the invention are represented by the formula:
R¹-Phe-Pro¹-AA²-Gly-AA₍ₙ₎-AA³-NH₂
wherein R¹ is preferably a halogen atom, preferably a fluorine or chlorine atom, a carboxyl group, an amino group or a nitro group, with all modifications preferably at the C4 atom of Phe; Pro¹ is 3,4-dehydro Pro, Homo-Pro, cis- or trans-4OH-Pro or Pro, as listed in order of preference, AA² is preferably Arg, Ile, Leu or His, with Arg being especially preferred; AA.sub.(n) is 0-2 amino acid residues, if n=1, then Gly is preferred and if n=2, then Ile-Gly, Ile-Ile or Gly-Gly is preferred; AA³ is preferably Trp or Gly, with Trp most preferred.

Also within the scope of the present invention are combinations of any of the peptides of formula (1) through formula (13) and the dermatological use of such combinations. Also included are chemically combined polypeptides formed by combining two or more of the peptides disclosed herein. Such chemically combined polypeptides preferably comprise from at least about three to at least about ten modified and/or unmodified amino acids.

The groupings of the peptides of the invention into the formulas described herein are provided only as a matter of convenience and should not be considered limiting in any manner.

In one embodiment, the peptides are tripeptides characterized either by optional replacement of the Leu residue of Pro-Leu-Gly-NH₂ with an amino acid selected from the group of Trp, Orn, Lys, Arg, D-Arg, or Ile; optional replacement of the Pro residue with dehydro-Pro, preferably 3,4-dehydro-Pro; optional modification of the carboxyl terminus amide group with a substituent selected from a carboxyl group, an hydroxyalkyl group, preferably a hydroxymethyl group, an alkoxycarbonyl group, or an alkylated carbamyl group; optional modification of the amino terminus heterocyclic group or dehydro-heterocyclic group with a substituent selected from the group of a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group; or an alkylamino group or a dialkylamino group, preferably a methyl or ethylamino or a dimethyl or diethylamino group; and/or optional modification of the hydrogen atoms at the nitrogen atoms of the amino acid peptide bonds with a lower alkyl group, preferably having 1 to 3 carbon atoms.

Tripeptides or pharmaceutically acceptable salts thereof can be represented by formula (1):
R¹-Pro¹-AA¹-NR²—CH₂—R (1)
where Pro¹ represents the amino acid Pro or dehydro-Pro, preferably 3,4-dehydro Pro; AA¹ represents an amino acid of the group of Trp, Orn, Lys, Leu, Arg, D-Arg, or Ile; R represents a carboxyl group, a hydroxyalkyl group, a carbamyl group, an alkylcarbamyl group, or an alkoxycarbonyl group; R¹ represents a hydrogen atom, a lower alkyl group, preferably having 1 to 3 carbon atoms, a halogen atom, preferably a fluorine or chlorine atom, a hydroxyl group, sulphydryl group, or an alkylamino or dialkylamino group, preferably a methyl or ethylamino group or dimethyl or diethylamino group; and, R² represents a hydrogen atom or a lower alkyl group, preferably having 1 to 3 carbon atoms, with the proviso that where Pro¹ is Pro and AA¹ is Leu, then R¹ and R² are not both hydrogen when R is a carbamyl (amide) group.

Some preferred tripeptides of formula (1) which may be utilized alone or in combination with other peptides disclosed herein are characterized by replacement of Leu, and are further characterized by having the N-terminus Pro¹ residue and C-terminus amide group remain unmodified, which can be represented by formula (1a). Formula (1a) is depicted as:
Pro¹-AA¹-Gly-NH₂ (1a)
wherein Pro¹ and AA¹ are as described above for formula (1). The tripeptides of formula (1a), may be utilized alone or in combination with other peptides disclosed herein. Examples of tripeptides of formula (1a) are: Pro-Trp-Gly-NH₂; Pro-Arg-Gly-NH₂; Pro-D-Arg-Gly-NH₂; Pro-Lys-Gly-NH₂ ; Pro-O r n-Gly-NH₂; and Pro-Ile-Gly-NH₂.

A second group of tripeptides of formula (1) which may be utilized alone or in combination with other peptides disclosed herein are characterized by optional replacement of Leu, and are further characterized by optional modification of the N-terminus heterocyclic nitrogen ring of Pro¹, preferably at the C-4 position of the heterocyclic nitrogen ring, and particularly preferably by addition of a cis- or trans-hydroxyl group or a cis- or trans-sulphydryl group at the C4 position, and wherein the C-terminus amide group preferably remains unmodified, which can be represented by formula (1b):
R¹-Pro¹-AA¹-Gly-NH₂ (1b)
wherein Pro¹, AA¹ and R¹ are as described above for formula (1). Examples of tripeptides of formula (1b) are: cis- or trans-4-OH-Pro-D-Arg-Gly-NH₂; cis- or trans-4-OH-Pro-Ile-Gly-NH₂; cis- or trans-4-OH-Pro-Arg-Gly-NH₂; cis- or trans-4-OH-Pro-Trp-Gly-NH₂; and cis- or trans-4-thio-Pro-Leu-Gly-NH₂.

A third group of preferred compositions of the tripeptides of formula (1) which may be utilized alone or in combination with other peptides disclosed herein are characterized by optional replacement of Leu, optional modification of the C-terminus amide group, optional modification of the C-terminus hydrogen atom at the nitrogen comprising the peptide bond between Leu-Gly, and by having the N-terminus heterocyclic nitrogen ring of Pro¹ remain unmodified, which can be represented by formula (1c). Formula (1c) is depicted as:
Pro¹-AA¹-NR²—CH₂—R (1c)
wherein Pro¹, AA¹, and R and R² are as described above for formula (1), with the proviso that where Pro¹ is Pro and AA¹ is Leu, R² cannot be hydrogen when R is either a carboxyl group or a hydroxyalkyl group, and with the further proviso that when Pro¹ is Pro¹ and AA¹ is Trp, R² is not hydrogen when R is a hydroxyalkyl group. Examples of tripeptides of formula (1c) are: Pro-Leu-N(CH₃)CH₂CONH₂ and Pro-Trp-NHCH₂—CO₂H.

In another embodiment of the invention, additional tripeptides are characterized by replacement of Leu with Arg or D-Arg; replacement of Gly with Ala; optional replacement of Pro with dehydro-Pro, preferably 3,4-dehydro-Pro; optional modification of the C-terminus amide group with a functional group selected from a carboxyl group, a hydroxyalkyl group, preferably a hydroxymethyl group, an alkoxycarbonyl group, and an alkylated carbamyl group; optional modification of the N-terminus heterocyclic nitrogen ring of Pro¹ with a substituent selected from a lower alkyl group preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group; and an alkylamino group or a dialkylamino group, preferably a methyl or ethyl amino or dimethyl or diethyl amino group; and/or optional modification of the hydrogen atoms at the nitrogen atoms of the amino acid peptide bonds with a lower alkyl group, preferably having 1 to 3 carbon atoms. This embodiment includes peptides represented by the following formula (2):
R¹-Pro¹-AA¹-Ala-R (2)
and pharmaceutically acceptable salts thereof, where Pro¹ represents the amino acid Pro or dehydro-Pro, preferably 3,4-dehydro Pro; AA¹ represents an amino acid of the group of Arg or D-Arg; R represents a carboxyl group, a hydroxyalkyl group, a carbamyl group, an alkylcarbamyl group, or an alkoxycarbonyl; and, R¹ represents a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

Exemples of tripeptides of formula (2) which may be utilized alone or in combination with other peptides disclosed herein are characterized by replacement of the Leu and Gly in Pro-Leu-Gly-NH₂, and by the N-terminus Pro¹ residue and C-terminus amide remain unmodified, which can be represented by formula (2a). Formula (2a) is depicted as:
R¹-Pro¹-AA¹-Ala-NH₂ (2a)
wherein Pro¹ and AA¹ are as described above for formula (2). Examples of tripeptides of formula (2a) are: Pro-Arg-Ala-NH₂ and Pro-D-Arg-Ala-NH₂.

In further embodiments, the small tripeptides are characterized by replacement of Leu with Orn; replacement of Gly with Tyr; optional replacement of Pro with dehydro-Pro, preferably 3,4-dehydro-Pro; optional modification of the C-terminus amide group with a substituent selected from the group of a carboxyl group, a hydroxyalkyl group, preferably hydroxymethyl, an alkoxycarbonyl group, or an alkylated carbamyl group; optional modification of the N-terminus heterocyclic nitrogen ring of Pro¹ with a substituent selected from lower alkyl groups, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino group or a dialkylamino group, preferably a methyl or ethylamino or a dimethyl or diethylamino group; and/or optional modification of the hydrogen atoms at the nitrogen atoms of the amino acid peptide bonds with a lower alkyl group, preferably having 1 to 3 carbon atoms. Such tripeptides or pharmaceutically acceptable salts thereof can be represented by formula (3):
R¹-Pro¹-AA¹-Tyr-R (3)
where Pro¹ represents the amino acid Pro or dehydro-Pro, preferably 3,4-dehydro Pro; AA¹ represents the amino acid Orn; R represents a carboxyl group, a hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or a dimethyl or diethylamino group.

The following paragraphs disclose compositions of the tripeptides of formula (3), which may be utilized alone or in combination with other peptides disclosed herein.

Tripeptides of formula (3) that may be utilized alone or in combination with other peptides disclosed herein are characterized by replacement of Leu and Gly, optional modification of the N-terminus heterocyclic nitrogen ring of Pro¹, and by having the C-terminus amide remain unmodified, can be represented by formula (3a):
R¹-Pro¹-AA¹-Tyr-NH₂ (3)
where Pro¹, AA¹ and R¹ are as described for formula (3). Examples of tripeptides of formula (3a) are: Pro-Orn-Tyr-NH₂ and cis- or trans-4-OH-Pro-Orn-Tyr-NH₂.

In yet another embodiment, the peptides are tetrapeptides characterized by either addition of a C-terminus amino acid of Trp or Tyr to Gly or addition of a N-terminus amino acid of Trp or Phe to Pro to Pro-Leu-Gly-NH₂ ; optional replacement of Leu with Ile, Arg, D-Arg, or Trp; optional replacement of Pro with dehydro-Pro, preferably 3,4-dehydro-Pro; optional modification of the C-terminus amide with a substituent selected from the group of a carboxyl group, a hydroxyalkyl group, preferably a hydroxymethyl group, an alkoxycarbonyl group, or an alkylated carbamyl group; optional modification of the heterocyclic nitrogen rings of Pro¹ and Trp and optional modification of the aromatic ring of Phe with a substituent selected from the group of a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or a dialkylamino group, preferably a methyl or ethylamino or a dimethyl or diethylamino group; and/or optional modification of the hydrogen atoms at the nitrogen atoms of the amino acid peptide bonds with a lower alkyl group, preferably having 1 to 3 carbon atoms.

One embodiment of the tetrapeptides or pharmaceutically acceptable salts thereof including a C-terminus amino acid addition can be represented by the formula (4):
R¹-Pro¹-AA¹-Gly-AA²-R (4)
where Pro¹ represents the amino acid Pro or dehydro-Pro, preferably 3,4-dehydro Pro; AA¹ represents an amino acid of the group of Ile, Leu, Arg, D-Arg or Trp; AA² represents Trp or Tyr; R represents a carboxyl group, hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a dehydro group, a halogen atom, preferably a fluorine or chlorine atom, a hydroxyl group, a sulphydryl group, or an alkylamino or dialkylamino group, preferably a methyl or ethylamino or dimethyl or diethylamino group.

Examples of tetrapeptides of formula (4) that may be utilized alone or in combination with other peptides disclosed herein are characterized by addition of Trp or Tyr to the C-terminus Gly, by optional replacement of Leu, by optional modification of the N-terminus heterocyclic nitrogen ring of Pro¹, and by having the C-terminus amide remain unmodified, which can be represented by formula (4a):
R¹-Pro¹-AA¹-Gly-AA²-NH₂ (4a)
wherein Pro¹, AA¹, AA², and R¹ are as described for formula (4). Examples of tetrapeptides of formula (4a) are: cis- or trans-4-OH-Pro-Leu-Gly-Trp-NH₂ (SEQ ID NO: 1); cis- or trans-4-OH-Pro-Leu-Gly-Trp-NH₂; (SEQ ID NO: 2); cis- or trans-4-OH-Pro-Ile-Gly-Trp-NH₂; cis- or trans-4-OH-Pro-D-Arg-Gly-Trp-NH₂; and 3,4-dehydro-Pro-D-Arg-Gly-Trp-NH₂.

Another example of a tetrapeptide of formula (4a) is 3,4-dehydro-Pro-Arg-Gly-Trp-NH₂ (SEQ ID NO: 62)

Other tetrapeptides of formula (4) that may be utilized alone or in combination with other peptides disclosed herein to treat patients suffering from physiological, psychosomatic, neurological or psychiatric disorders are characterized by addition of Trp or Tyr to the C-terminus Gly, by optional replacement of Leu, and by having the N-terminus heterocyclic nitrogen ring of Pro¹ remain unmodified, which can be represented by formula (4b):
Pro¹-AA¹-Gly-AA²-NH₂ (4b)
wherein Pro¹, AA¹ and AA² are as described for formula (4). Examples of tetrapeptides of formula (4b) are: Pro-Ile-Gly-Trp-NH₂ (SEQ ID NO: 3); 3,4-dehydro-Pro-Ile-Gly-Trp-NH₂ (SEQ ID NO: 4); Pro-Leu-Gly-Trp-NH₂ (SEQ ID NO: 5); Pro-Leu-Gly-Tyr-NH₂ (SEQ ID NO: 6); Pro-Arg-Gly-Trp-NH₂ (SEQ ID NO: 7); Pro-Trp-Gly-Trp-NH₂ (SEQ ID NO: 8); Pro-D-Arg-Gly-Trp-NH₂; and Pro-Ile-Gly-Tyr-NH₂. (SEQ ID NO: 9)

Another embodiment of the tetrapeptides or pharmaceutically acceptable salt thereof including a N-terminus amino acid addition can be represented by formula (5):
R¹-AA¹-R²-Pro¹-AA²-Gly-R (5)
where Pro¹ represents the amino acid Pro or dehydro-Pro, preferably 3,4-dehydro Pro; AA¹ represents an amino acid of the group of Trp, Tyr, or Phe; AA² represents an amino acid of the group of Leu, Ile, or Trp; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, a sulphydryl group, or an alkylamino or dialkylamino group, preferably a methyl or ethylamino or dimethyl or diethylamino group.

Tetrapeptides of formula (5) may be used in combination with one or more other peptides disclosed herein.

Examples of tetrapeptides of formula (5) that may be utilized alone or in combination with other peptides disclosed herein are characterized by addition of Trp, Tyr, or Phe to the N-terminus Pro¹, optional replacement of Leu, optional modification of the heterocyclic nitrogen rings of Pro¹ and Trp and optional modification of the aromatic ring of Phe and Tyr, and wherein the C-terminus amide remains unmodified, which can be represented by formula (5a):
R¹-AA¹-R²-Pro¹-AA²-Gly-NH₂ (5a)
wherein Pro¹, AA¹, AA², R¹, and R² are as described for formula (5), with the proviso that where Pro¹ is Pro, R¹ and R² cannot both be a hydrogen atom when AA¹ is Tyr and AA² is Trp, since Tyr-Pro-Trp-Gly-NH₂ (SEQ ID NO: 54 is a known compound, and with the further proviso that where Pro¹ is Pro and AA² is Leu, R¹ and R² cannot both be a hydrogen atom when AA¹ is Phe or Tyr. Examples of tetrapeptides of formula (5a) are: Trp-Pro-Leu-Gly-NH₂ (SEQ ID NO: 10); Phe-Pro-Leu-Gly-NH₂ (SEQ ID NO: 11); 4-F-Phe-Pro-Leu-Gly-NH₂ (SEQ ID NO: 12); 4-Cl-Phe-Pro-Leu-Gly-NH₂ (SEQ ID NO: 13); 4-F-Phe-Pro-Ile-Gly-NH₂ (SEQ ID NO: 14); 4-F-Phe-cis- or trans-4-OH-Pro-Leu-Gly-NH₂ (SEQ ID NO: 15); 4-F-Phe-cis- or trans-4-OH-Pro-Ile-Gly-NH₂ (SEQ ID NO: 16); Trp-Pro-Leu-Gly-NH₂ (SEQ ID NO: 17); Trp-Pro-Ile-Gly-NH₂ (SEQ ID NO: 18); Trp-cis- or trans-4-OH-Pro-Leu-Gly-NH₂ (SEQ ID NO: 19); and Trp-cis- or trans-4-OH-Pro-Ile-Gly-NH₂ (SEQ ID NO: 20).

Another example of a tetrapeptide of formula (5a) is 4-Cl-Phe-cis- or trans-4-OH-Pro-Ile-Gly-NH₂ (SEQ ID NO: 60).

Other tetrapeptides of formula (5) that may be utilized alone or in combination with other peptides disclosed herein are characterized by addition of Phe to the N-terminus Pro¹ optional replacement of Leu with Arg, optional modification of the heterocyclic nitrogen ring of Pro¹ and optional modification of the aromatic ring of Phe, and modification of the C-terminus amide to a carbamyl group, which can be represented by formula (5b):
R¹-AA¹-R²-Pro¹-AA²-Gly-R (5b)
wherein Pro¹, AA¹, AA², R¹, R² and R are as described for formula (5). Examples of tetrapeptides of formula (5b) include, but are not solely limited to, additional optional modification of the N-terminus heterocyclic nitrogen ring of Pro¹ with a cis- or trans-4-OH— group; and additional optional modifications at AA², preferably Arg. A preferred peptide of formula (5b) is 4-F-Phe-cis- or trans-4-OH-Pro-Arg-Gly-1,2,3,4-Tetrahydroisoquinoline-3-carboxamide (SEQ ID NO: 75).

In yet another embodiment of the invention, the peptides are pentapeptides with either addition of two N-terminus amino acids of Phe, Tyr, Leu, or Ile to Pro¹, addition of a N-terminus amino acid of Phe or Tyr to Pro¹ and a C-terminus amino acid addition of Trp to Gly, or addition of a C-terminus amino acids of Trp to Gly and an internal amino acid between Pro¹ and Gly, to Pro-Leu-Gly-NH₂ ; optional replacement of Leu with Ile or Trp; optional replacement of Pro with dehydro-Pro, preferably 3,4-dehydro-Pro; optional modification of the C-terminus amide with a substituent selected from the group of a carboxyl group, a hydroxyalkyl group, preferably a hydroxymethyl group, an alkoxycarbonyl group, or an alkylated carbamyl group; optional modification of the heterocyclic nitrogen ring of Pro¹ and optional modification of the aromatic rings of Tyr or Phe with a substituent selected from the group of a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or a dialkylamino group, preferably a methyl or ethylamino or a dimethyl or diethylamino group; and/or optional modification of the hydrogen atoms at the nitrogen atoms of the amino acid peptide bonds with a lower alkyl group, preferably having 1 to 3 carbon atoms.

One embodiment of the pentapeptides including addition of two N-terminus amino acids or pharmaceutically acceptable salt thereof can be represented by formula (6):
R¹-AA¹-AA²-R²-Pro¹-AA³-Gly-R (6)
where Pro¹ represents the amino acid Pro or dehydro-Pro, preferably 3,4-dehydro Pro; AA¹ and AA² each independently represent an amino acid of the group of Phe or Tyr; AA³ represents an amino acid of the group of Leu or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

Examples of pentapeptides of formula (6) that may be utilized alone or in combination with other peptides disclosed herein to treat patients suffering from physiological, psychosomatic, neurological or psychiatric disorders are characterized by addition of two N-terminus amino acids of Phe and Tyr to Pro¹, optional modification of the heterocyclic nitrogen ring of Pro¹ and optional modification of the aromatic rings of Phe or Tyr, optional replacement of Leu, and by having the C-terminus amide of Gly remain unmodified, which can be represented by formula (6a):
R¹-AA¹-AA²-R²-Pro¹-AA³-Gly-NH₂ (6a)
wherein Pro¹, AA¹, AA², R¹, and R² are as described for formula (6). Examples of pentapeptides of formula (6a) are: 4-F-Phe-Tyr-Pro-Leu-Gly-NH₂ (SEQ ID NO: 21); 4-Cl-Phe-Tyr-Pro-Leu-Gly-NH₂ (SEQ ID NO: 22); Phe-Tyr-Pro-Leu-Gly-NH₂ (SEQ ID NO: 23); Phe-Tyr-Pro-Ile-Gly-NH₂ (SEQ ID NO: 24); Phe-Tyr-cis- or trans-4-OH-Pro-Leu-Gly-NH₂ (SEQ ID NO: 25); Phe-Tyr-cis- or trans-4-OH-Pro-Ile-Gly-NH₂ (SEQ ID NO: 26); Tyr-Tyr-Pro-Leu-Gly-NH₂ (SEQ ID NO: 27); Tyr-Tyr-Pro-Ile-Gly-NH₂ (SEQ ID NO: 28); Tyr-Tyr-cis- or trans-4-OH-Pro-Leu-Gly-NH₂ (SEQ ID NO: 29); and, Tyr-Tyr-cis- or trans-4-OH-Pro-Ile-Gly-NH₂ (SEQ ID NO: 30).

Another embodiment of the invention provides pentapeptides or pharmaceutically acceptable salts thereof including addition of both a N-terminus amino acid and a C-terminus amino acid represented by formula (7):
R¹-AA¹-R²-Pro¹-AA²-Gly-AA³-R (7)
where Pro¹ represents the amino acid Pro or dehydro-Pro, preferably 3,4-dehydro Pro; AA¹ represents an amino acid of the group of Phe or Tyr; AA² represents an amino acid of the group of Leu, Ile, Arg, D-Arg, or Trp; AA³ represents the amino acid Trp; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

Examples of pentapeptides of formula (7) which may be utilized alone or in combination with other peptides disclosed herein are characterized by addition of a N-terminus amino acid of Phe or Tyr to Pro¹, addition of a C-terminus amino acid of Trp to Gly, optional modification of the heterocyclic nitrogen ring of Pro¹ and optional modification of the aromatic rings of Phe or Tyr, optional replacement of Leu with Ile, Arg, D-Arg, or Trp, and the C-terminus amide remaining unmodified, and can be represented by formula (7a):
R¹-AA¹-R²-Pro¹-AA²-Gly-Trp-NH₂ (7a)
wherein Pro¹, AA¹, AA², R¹, and R² are as described for formula (7). Examples of pentapeptides of formula (7a) are: Phe-Pro-Leu-Gly-Trp-NH₂ (SEQ ID NO: 31); Tyr-Pro-Leu-Gly-Trp-NH₂ (SEQ ID NO: 32); Phe-cis- or trans-4-OH-Pro-Leu-Gly-Trp-NH₂ (SEQ ID NO: 33); Phe-Pro-Ile-Gly-Trp-NH₂ (SEQ ID NO: 34); Phe-cis- or trans-4-OH-Pro-Ile-Gly-Trp-NH₂ (SEQ ID NO: 35); Tyr-cis- or trans-4-OH-Pro-Leu-Gly-Trp-NH₂ (SEQ ID NO: 36); Tyr-Pro-Ile-Gly-Trp-NH₂ (SEQ ID NO: 37); Tyr-cis- or trans-4-OH-Pro-Leu-Gly-Trp-NH₂ (SEQ ID NO: 38); Tyr-Pro-Trp-Gly-Trp-NH₂ (SEQ ID NO: 39); Tyr-cis- or trans-4-OH-Pro-Trp-Gly-Trp-NH₂ (SEQ ID NO: 40); 4-F-Phe-cis- or trans-4-OH-Pro-Ile-Gly-Trp-NH₂ (SEQ ID NO: 41); 4-F-Phe-cis- or trans-4-OH-Pro-Leu-Gly-Trp-NH₂ (SEQ ID NO: 42); 4-F-Phe-cis- or trans-4-OH-Pro-Arg-Gly-Trp-NH₂ (SEQ ID NO: 43); and 4-F-Phe-cis- or trans-4-OH-Pro-D-Arg-Gly-Trp-NH₂.

Additional examples of pentapeptides of formula (7a) include: 3-F-Phe-cis- or trans-4-OH-Pro-Arg-Gly-Trp-NH₂ (SEQ ID NO: 66); 2-F-Phe-cis- or trans-4-OH-Pro-Arg-Gly-Trp-NH₂ (SEQ ID NO: 68); and 4-Cl-Phe-cis- or trans-4-OH-Pro-Arg-Gly-Trp-NH₂ (SEQ ID NO: 61).

Another group of pentapeptides of formula (7a) is characterized by the optional modification of Pro¹ to dehydro-Pro, preferably 3,4-dehydro-Pro, and includes: 4-F-Phe-3,4-dehydro-Pro-Ile-Gly-Trp-NH₂ (SEQ ID NO: 72); and 4-F-Phe-3,4-dehydro-Pro-Arg-Gly-Trp-NH₂ (SEQ ID NO: 55).

Further embodiments encompassed within formula (7a) include compounds having additional optional modifications at AA², preferably Arg, His, Homo-Arg, L-Allo-Ile, or canavanine; additional optional modifications at R¹ and/or R² (preferably R¹) and preferably an amino group, a carboxyl group, a nitro group, or a phosphono group (preferably as phosphono-Tyr); additional optional modification of the heterocyclic nitrogen ring of Pro¹, preferably cis- or trans-4-OH or Homo-Pro. Additional preferred peptides of formula (7a) are: 4-NH₂-Phe-cis- or trans-4-OH-Pro-Arg-Gly-Trp-NH₂ (SEQ ID NO: 63); 4-F-Phe-cis- or trans-4-OH-Pro-His-Gly-Trp-NH₂ (SEQ ID NO: 64); 4-NO₂-Phe-cis- or trans-4-OH-Pro-Arg-Gly-Trp-NH₂ (SEQ ID NO: 65); 4-CH₃ O-Phe-cis- or trans-4-OH-Pro-Arg-Gly-Trp-NH₂ (SEQ ID NO: 59); 4-F-Phe-cis- or trans-4-OH-Pro-Homo-Arg-Gly-Trp-NH₂ (SEQ ID NO: 71); 4-F-Phe-Homo-Pro-Ile-Gly-Trp-NH₂ (SEQ ID NO: 69); 4-F-Phe-Homo-Pro-Arg-Gly-Trp-NH₂ (SEQ ID NO: 57); and 4-F-Phe-cis- or trans-4-OH-Pro-L-Allo-Ile-Gly-Trp-NH₂. (SEQ ID NO: 73).

Examples of pentapeptides of formula (7) which may be utilized alone or in combination with other peptides disclosed herein to treat patients suffering from physiological, psychosomatic, neurological or psychiatric disorders are characterized by addition of an N-terminus amino acid of Phe; addition of a C-terminus amino acid of Trp to Gly; optional modification of the heterocyclic nitrogen ring of Pro¹ and optional modification of the aromatic ring of Phe; and by the C-terminus amide remaining unmodified, and can be represented by formula (7b):
R¹-AA¹-R²-Pro¹-AA²-Gly-Trp-NH₂ (7b)
wherein AA¹ is Phe; and Pro¹, AA², R¹ and R² are as described for formula (7) with the following additional optional modification of the N-terminus heterocyclic nitrogen ring of Pro¹ with a substituent selected from the group consisting of cis-4-OH—, trans-4-OH—, cis-3-OH—, and trans-3-OH—; and additional optional modifications at R¹, preferably two or more halogen atoms or a cyano group. Examples of pentapeptides of formula (7b) include: 3,4-Dichloro-Phe-cis- or trans-4-OH-Pro-Arg-Gly-Trp-NH₂ (SEQ ID NO: 76); 4-NC-Phe-cis- or trans-4-OH-Pro-Arg-Gly-Trp-NH₂ (SEQ ID NO: 77); 4-F-Phe-cis- or trans-4-OH-Pro-D-Leu-Gly-Trp-NH₂ (SEQ ID NO: 78); and 4-F-Phe-trans-3-Hydroxy-Pro-Arg-Gly-Trp-NH₂ (SEQ ID NO: 79).

Additional examples of pentapeptides of formula (7) that may be utilized alone or in combination with other peptides disclosed herein are characterized by addition of an N-terminus amino acid of Phe, addition of a C-terminus amino acid of Trp to Gly, optional modification of the heterocyclic nitrogen ring of Pro¹ and optional modification of the aromatic ring of Phe, and the absence of the C-terminus amide, which can be represented by formula (7c):
R¹-AA¹-R²-Pro¹-AA²-Gly-Trp (7c)
where Pro¹, AA¹, AA², R¹ and R² are as described for formula (7) with additional optional modifications at AA², preferably Homo-Arg; and additional optional modification of the N-terminus heterocyclic nitrogen ring of Pro¹ with a substituent selected from the group consisting of cis-4-OH—, trans-4-OH—, cis-3-OH— and trans-3-OH. A preferred peptide of formula (7c) is 4-F-Phe-cis- or trans-4-OH-Pro-Homo-Arg-Gly-Trp (SEQ ID NO: 74).

Another group of pentapeptides of formula (7) that may be utilized alone or in combination with other peptides disclosed herein are characterized by addition of an N-terminus amino acid of Phe, Tyr or PhenylGly; addition of a C-terminus amino acid of Trp or AzaTrp to Gly; optional modification of the N-terminus heterocyclic nitrogen ring of Pro¹ and optional modification of the aromatic rings of Phe, Tyr and PhenylGly; and additional optional modification at R, preferably a hydroxyamino group, which can be represented by formula (7d):
R¹-AA¹-R²-Pro¹-AA²-Gly-AA³-R (7d)
wherein Pro¹, AA¹, AA², AA³, R¹, R² and R are as described for formula (7) with the following additional optional modifications at Pro¹, preferably Homo-Pro; additional optional modifications at AA¹, preferably PhenylGly; additional optional modifications at AA³, preferably AzaTrp; additional optional modifications at R¹, preferably two or more halogen atoms, a haloform, or a methoxyl group; additional optional modifications at R², preferably a cis- or trans-3-OH— group; and additional optional modifications at R, preferably a hydroxyamino group. Examples of pentapeptides of formula (7d) include: 4-CH₃ O-Phe-3,4-Dehydro-Pro-Arg-Gly-Trp-NH₂ (SEQ ID NO: 81); 2,4-Di-F-Phe-3,4-Dihydro-Pro-Arg-Gly-Trp-NH₂ (SEQ ID NO: 82); 4-CF₃-Phe-3,4-Dehydro-Pro-Arg-Gly-Trp-NH₂ (SEQ ID NO: 83); 4-F-PhenylGly-3,4-Dehydro-Pro-Arg-Gly-Trp-NH₂ (SEQ ID NO: 84); 3-F-Tyr-3,4-Dehydro-Pro-Arg-Gly-Trp-NH₂ (SEQ ID NO: 85); 4-F-Phe-3,4-Dehydro-Pro-Arg-Gly-Trp-NHOH (SEQ ID NO: 86); 3,4-Di-Cl-Phe-3,4-Dihydro-Pro-Arg-Gly-Trp-NH₂ (SEQ ID NO: 87); 2-F-Tyr-3,4-Dehydro-Pro-Arg-Gly-Trp-NH₂ (SEQ ID NO: 88); and 4-F-Phe-3,4-Dehydro-Pro-Arg-Gly-7-AzaTrp-NH₂ (SEQ ID NO: 89).

Another group pentapeptides of formula (7) which may be utilized alone or in combination with other peptides disclosed herein are characterized by addition of an N-terminus amino acid of Phe; addition of a C-terminus amino acid of Trp to Gly; optional modification of the heterocyclic nitrogen ring of Pro¹ and optional modification of the aromatic rings of Phe and Trp; and by having the C-terminus amide remain unmodified, which can be represented by formula (7e):
R¹-AA¹-R²-Pro¹-AA²-Gly-R⁴-Trp-NH₂ (7e)
wherein Pro¹, AA¹, AA², R¹ and R² are as described for formula (7) with the following additional optional modification of the N-terminus heterocyclic nitrogen ring of Pro¹ with a substituent selected from the group consisting of cis-4-OH—, trans-4-OH—, cis-3-OH—, and trans-3-OH—; and R⁴ represents a modification of the tryptophan residue at one of C4, C5, C6 and C7 with a halogen atom, a hydroxyl group, or an alkyl group. Examples of pentapeptides of formula (7e) include: 4-F-Phe-cis- or trans-4-OH-Pro-Arg-Gly-4-F-Trp-NH₂ (SEQ ID NO: 107) and 4-F-Phe-cis- or trans-4-OH-Pro-Arg-Gly-7-Methyl-Trp-NH₂ (SEQ ID NO: 108).

Another group of pentapeptides of formula (7) which may be utilized alone or in combination with other peptides disclosed herein are characterized by addition of an N-terminus amino acid of Phe; addition of a C-terminus amino acid of Trp to Gly; optional modification of the heterocyclic nitrogen ring of Pro¹ and optional modification of the aromatic rings of Phe and Leu; and by having the C-terminus amide remain unmodified, which can be represented by formula (7f):
R¹-AA¹-R²-Pro¹-R⁵-AA²-Gly-Trp-NH₂ (7f)
wherein Pro¹, AA¹, AA², R¹ and R² are as described for formula (7) with the following additional optional modification of the N-terminus heterocyclic nitrogen ring of Pro¹ with a substituent selected from the group consisting of cis-4-OH—, trans-4-OH—, cis-3-OH—, and trans-3-OH—; and R⁵ represents at least one halogen atom. A preferred composition of the pentapeptides of formula (7f) is 4-F-Phe-4-OH-Pro-5,5,5-Trifluoro-Leu-Gly-Trp-NH₂ (SEQ ID NO: 109).

Another group of pentapeptides of formula (7) which may be utilized alone or in combination with other peptides disclosed herein are characterized by addition of an N-terminus amino acid of Phe; addition of a C-terminus amino acid of Trp to Gly; additional optional modifications at Pro¹, preferably Homo-Pro; optional modification of the heterocyclic nitrogen ring of Pro¹ and optional modification of the aromatic rings of Phe and Trp; and by having the C-terminus amide remain unmodified, which can be represented by formula (7g):
R¹-AA¹-R²-Pro¹-AA²-Gly-R⁴-AA³-R (7g)
wherein Pro¹, AA¹, AA², AA³, R¹, R² and R are as described for formula (7) with the following additional optional modifications at Pro¹, preferably Homo-Pro or 3,4-dihydro-Pro; additional optional modifications at R², preferably cis- or trans-3-OH— group; and R⁴ represents a halogen atom, a methyl group, a methoxyl group or a hydroxyl group. Examples of pentapeptides of formula (7g) include: 4-F-Phe-3,4-Dehydro-Pro-Arg-Gly-4-F-Trp-NH₂ (SEQ ID NO: 90); 4-F-Phe-3,4-Dehydro-Pro-Arg-Gly-5-F-Trp-NH₂ (SEQ ID NO: 91); 4-F-Phe-3,4-Dihydro-Pro-Arg-Gly-6-F-Trp-NH₂ (SEQ ID NO: 92); 4-F-Phe-3,4-Dehydro-Pro-Arg-Gly-3-CH₃ O-Trp-NH₂ (SEQ ID NO: 93); 4-F-Phe-3,4-Dehydro-Pro-Arg-Gly-N-Methyl-Trp-NH₂ (SEQ ID NO: 94); 4-F-Phe-3,4-Dehydro-Pro-Arg-Gly-1-Methyl-Trp-NH₂ (SEQ ID NO: 95); 4-F-Phe-3,4-Dehydro-Pro-Arg-Gly-4-Methyl-Trp-NH₂ (SEQ ID NO: 96); 4-F-Phe-3,4-Dehydro-Pro-Arg-Gly-5-Methyl-Trp-NH₂ (SEQ ID NO: 97); 4-F-Phe-3,4-Dehydro-Pro-Arg-Gly-6-Methyl-Trp-NH₂ (SEQ ID NO: 98); and 4-F-Phe-3,4-Dehydro-Pro-Arg-Gly-5-Hydroxy-Trp-NH₂ (SEQ ID NO: 99).

In yet another embodiment of the invention, pentapeptides or pharmaceutically acceptable salts thereof including addition of a C-terminus amino acid and an internal amino acid can be represented by the following formula (8):
R¹-Pro¹-AA¹-AA²-Gly-AA³-R (8)
where Pro¹ represents the amino acid Pro or dehydro-Pro, preferably 3,4-dehydro-Pro; AA¹ and AA² each independently represent an amino acid of the group of Leu or Ile; AA³ represents Trp; R represents a carboxyl group, a hydroxyalkyl group, a carbamyl group, an alkylcarbamyl group, or an alkoxycarbonyl group; and, R¹ represents a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino group or dimethyl or diethylamino group.

Examples of pentapeptides of formula (8) which may be utilized alone or in combination with other peptides disclosed herein are characterized by addition of a C-terminus amino acid of Trp to Gly, addition of an internal amino acid of Leu or Ile between Pro¹ and Gly, optional modification of the heterocyclic nitrogen ring of Pro¹, optional replacement of Leu with Ile, and by having the C-terminus amide remain unmodified, which can be represented by formula (8a):
R¹-Pro¹-AA¹-AA²-Gly-Trp-NH₂ (8)
wherein Pro¹, AA¹, AA², and R¹ are as described for formula (8). Examples of pentapeptides of formula (8a) are: Pro-Ile-Leu-Gly-Trp-NH₂ (SEQ ID NO: 44) and cis- or trans-4-OH-Pro-Ile-Leu-Gly-Trp-NH₂ (SEQ ID NO: 45)

In another embodiment of the invention, pentapeptides or pharmaceutically acceptable salts thereof including addition of both a N-terminus amino acid and a C-terminus amino acid can be represented by the following formula (9):
R¹-AA¹-R²-Pro¹-AA²-Gly-AA³-R (9)
where Pro¹ represents the amino acid Pro or dehydro-Pro, preferably 3,4-dehydro Pro; AA¹ represents the amino acid Ala; AA² represents an amino acid of the group of Leu, Ile, Arg, D-Arg, Trp, or canavanine; AA³ represents the amino acid Trp; R represents a carboxyl group, hydroxyalkyl group, a carbamyl group, an alkylcarbamyl group, or an alkoxycarbonyl group; and, R¹ represents a pyridyl ring, preferably as a 3-(3-pyridyl) moiety; R² represents a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

An example of a peptide of formula (9) is 3-(3-pyridyl)-Ala-4-OH-Pro-Arg-Gly-Trp-NH₂ (SEQ ID NO: 70).

In another embodiment of the invention, hexapeptides or pharmaceutically acceptable salts thereof including addition of both a N-terminus amino acid and a C-terminus amino acid can be represented by formula (10):
R¹-AA¹-R²-Pro¹-AA²-AA⁴-Gly-AA³-R (10)
where Pro¹ represents the amino acid Pro or dehydro-Pro; AA¹ represents an amino acid of the group of Phe or Tyr; AA² represents an amino acid of the group of Leu, Ile, Arg, D-Arg, Trp, or canavanine; AA³ represents the amino acid Trp; AA⁴ represents the amino acid Gly or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group.

An example of a peptide of formula (10) is 4-F-Phe-3,4-Dehydro-Pro-Arg-Gly-Gly-Trp-NH₂ (SEQ ID NO: 80).

Examples of hexapeptides of formula (10) which may be utilized alone or in combination with other peptides disclosed herein are characterized by addition of a C-terminus amino acid of Trp, optional modification of the heterocyclic nitrogen ring of Pro¹, preferably a cis- or trans-4-OH group, a fluorine atom at position 4 of Phe; preferably Arg at AA²; Tpr at AA³; and Ile or Gly at AA⁴, and by having the C-terminus amide remain unmodified, which can be represented by formula (10a):
R¹-Phe-R²-Pro¹-AA²-AA⁴-Gly-Trp-NH₂ (10a)
wherein preferred peptides of formula (10a) are: 4-F-Phe-4-OH-Pro-Arg-Gly-Gly-Trp-NH₂ (SEQ ID NO: 58); and 4-F-Phe-4-OH-Pro-Arg-Ile-Gly-Trp-NH₂ (SEQ ID NO: 67).

In another embodiment of the invention, heptapeptides or pharmaceutically acceptable salts thereof including addition of both a N-terminus amino acid and a C-terminus amino acid can be represented by the following formula (11):
R¹-AA¹-R²-Pro¹-AA²-AA⁴-AA⁵-Gly-AA³-R (11)
where Pro¹ represents the amino acid Pro or dehydro-Pro; AA¹ represents an amino acid of the group of Phe or Tyr; AA represents an amino acid of the group of Leu, Ile, Arg, D-Arg, Trp, or canavanine; AA³ represents the amino acid Trp; AA⁴ and AA⁵ represent the amino acid Gly or Ile; R represents a carboxyl group, hydroxyalkyl group, a carbamyl group, an alkylcarbamyl group, or an alkoxycarbonyl group; and, R¹ and R² each independently represent a hydrogen atom, a lower alkyl group, preferably having 1 to 3 carbon atoms, a halogen atom, preferably a fluorine or chlorine atom, a hydroxyl group, preferably a cis- or trans-4-OH— group, a sulphydryl group, preferably a cis- or trans-4-thio- group, or an alkylamino or dialkylamino group, preferably a methyl or ethylamino or dimethyl or diethylamino group, or a phosphono group (preferably as phosphono-Tyr).

An example of a peptide of formula (11) is: 4-F-Phe-4-OH-Pro-Arg-Gly-Ile-Gly-Trp-NH₂ (SEQ ID NO: 56).

Examples of tetrapeptides of formula (12) or pharmaceutically acceptable salts thereof which may be utilized alone or in combination with other peptides disclosed herein are characterized by replacement of Pro with Arg; addition of an N-terminus amino acid of Phe to Arg; addition of a C-terminus amino acid of Trp to Gly; optional modification of the aromatic rings of Phe and by having the C-terminus amide remain unmodified, which can be represented by formula (12):
R¹-Phe-R²-Arg-Gly-Trp-NH₂ (12)
where R¹ represents a halogen atom and R² represents a carboxylic acid of a monocyclic organic compound with a three to six membered ring structure having a hetero nitrogen atom. Examples of pentapeptides of formula (12) include, but are not limited to: 4-F-Phe-isonipecotic acid-Arg-Gly-Trp-NH₂(4-pyridinecarboxylic acid) (SEQ ID NO: 100); 4-F-Phe-2-Carboxy-Azetidine-Arg-Gly-Trp-NH₂ (SEQ ID NO: 101); 4-F-Phe-2-carboxy-Aziridine-Arg-Gly-Trp-NH₂ (SEQ ID NO: 103); 4-F-Phe-3-Carboxy-1,4,5,6-Tetrahydropyridine-Arg-Gly-Trp-NH₂ (SEQ ID NO: 105); and 4-F-Phe-2-Carboxypyrrole-Arg-Gly-Trp-NH₂ (SEQ ID NO: 106).

The following paragraph discloses compositions of the pentapeptides of formula (13), which may be utilized alone or in combination with other peptides disclosed herein.

Examples of pentapeptides of formula (13) or a pharmaceutically acceptable salt thereof which may be utilized alone or in combination with other peptides disclosed herein are characterized by replacement of Pro with Arg; addition of an N-terminus amino acid of Phe to Arg; addition of an internal amino acid; addition of a C-terminus amino acid of Trp to Gly; optional modification of the aromatic ring of Phe and by C-terminus amide remaining unmodified, which can be represented by formula (13). Formula (13) is depicted as:
R¹-Phe-AA¹-Arg-Gly-Trp-NH₂ (13)
where AA¹ represents an amino acid selected from the group comprising 1-amino-1-carboxycyclopentane and 1-amino-1-carboxy-cyclopropyl and R¹ represents a halogen atom. Examples of pentapeptides of formula (13) include, but are not necessarily limited to, 4-F-Phe-1-Amino-1-Carboxycyclopentane-Arg-Gly-Trp-NH₂ (SEQ ID NO: 102) and 4-F-Phe-1-Amino-1-Carboxy-Cyclopropyl-Arg-Gly-Trp-NH₂ (SEQ ID NO: 104).

Gly in compounds of formula (7) through formula (11) may be replaced with Val or Ala. One peptide wherein Gly is substituted with Sar in formula (7) is 4-F-Phe-3,4-Dehydro-Pro-Arg-Sar-Trp-NH₂ (SEQ ID NO: 110).

In yet another embodiment of the invention, the peptides are polypeptides including chemical combinations and/or overlapping chemical combinations of any of the peptides of any of formula (1) through formula (I1) described above which may be utilized alone or in combination with other peptides disclosed herein. The chemical combinations and/or overlapping chemical combinations of the peptides disclosed preferably range from at least about three to at least about ten amino acids. Examples of such combinations include: 4-F-Phe-cis- or trans-4-OH-Pro-Ile-Gly-Trp-Gly-NH2 (SEQ ID NO: 46); 4-F-Phe-cis or trans-4-OH-Pro-Ile-Gly-Trp-Gly-Trp-NH₂ (SEQ ID NO: 47); 4-F-Phe-cis- or trans-4-OH-Pro-Leu-Gly-Trp-Gly-NH₂ (SEQ ID NO: 48); 4-F-Phe-cis- or trans-4-OH-Pro-Leu-Gly-Trp-Gly-Trp-NH₂ (SEQ ID NO: 49); Pro-Ile-Gly-Trp-Pro-Ile-Gly-NH₂ (SEQ ID NO: 50); 4-F-Phe-cis- or trans-4-OH-Pro-Arg-Gly-Trp-Gly-NH₂ (SEQ ID NO: 51); 4-F-Phe-cis or trans-4-OH-Pro-Arg-Gly-Trp-Gly-Trp-NH₂ (SEQ ID NO: 52); cis- or trans-4-OH-Pro-Ile-Gly-cis- or trans-4-OH-Pro-Ile-Gly-NH₂ (SEQ ID NO: 53); 3,4-dehydro-Pro-D-Arg-Gly-3,4-dehydro-Pro-D-Arg-Gly-NH₂; 3,4-dehydro-Pro-D-Arg-Gly-Trp-Gly-NH₂; and 3,4-dehydro-Pro-D-Arg-Gly-Trp-Gly-Trp-NH₂.

One group of tetrapeptides that may be used in accordance with the invention have the general formula:
R¹-Phe-Pro¹-AA²-AA³-NH₂
wherein R¹ is preferably a halogen atom, most preferably a fluorine or chlorine atom, a carboxyl group, an amino group or a nitro group, with all modifications preferably at the C4 atom of Phe; Pro¹ is 3,4-dehydro Pro, Homo-Pro, cis- or trans-4OH-Pro or Pro, as listed in order of preference, AA is preferably Ile, Leu or Arg; and AA³ is preferably Gly or Trp.

Another exemplary tetrapeptide of the present invention is Pro-Ile-Gly-Trp (SEQ ID NO: 3).

One goup of pentapeptides, hexapeptides and heptapeptides that may be used in accordance with the invention are represente by the formula:
R¹-Phe-Pro¹-AA²-Gly-AA₍ₙ₎-AA³-NH₂
wherein R¹ is preferably a halogen atom, preferably a fluorine or chlorine atom, a carboxyl group, an amino group or a nitro group, with all modifications preferably at the C4 atom of Phe; Pro¹ is 3,4-dehydro Pro, Homo-Pro, cis- or trans-4OH-Pro or Pro, as listed in order of preference, AA² is preferably Arg, Ile, Leu or His, with Arg being especially preferred; AA.sub.(n) is 0-2 amino acid residues, if n=1, then Gly is preferred and if n=2, then Ile-Gly, Ile-Ile or Gly-Gly is preferred; AA³ is preferably Trp or Gly, with Trp most preferred.

Without limitation, the peptides of this disclosure include MIF and peptides including the sequence prolyl-leucyl-glycinamide, Pro-Leu-Gly and peptides containing this sequence, peptides according to any of the peptide formulas of this disclosure, peptides listed in the appended Sequence Listing, peptides otherwise explicitly disclosed herein and any of the peptides disclosed herein by way of the incorporation by reference of related U.S. Pat. Nos. 6,767,897; 6,093,797; 5,767,083; and 5,589,460. Dermatologically and/or pharmaceutically acceptable salts of any of the peptides of this disclosure may also be used according to the invention.

Each of the patents, patent applications and other documents cited herein is hereby expressly incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A topical dermatological composition comprising:
a dermatologically acceptable vehicle; and
a pentapeptide of formula (7):
R¹-AA¹-R²-Pro¹-AA²-Gly-AA³-R (7)
wherein Pro¹ is Pro or dehydro-Pro and the N-terminus heterocyclic nitrogen ring of Pro¹ is optionally modified with a substituent selected from the group consisting of cis-4-OH—, trans-4-OH—, cis-3-OH— and trans-3-OH; AA¹ is Phe or Tyr; AA² is selected from the group consisting of Leu, Ile, Arg, D-Arg and Trp; AA³ is Trp; R is a group by which the carboxy-terminus of amino acid AA remains unmodified or is modified into a hydroxyalkyl group, a carbamyl group, an alkylcarbamyl group, or an alkoxycarbonyl group; and, R¹ and R² each independently represent a hydrogen atom, a lower alkyl group, a halogen atom, a hydroxyl group, a sulphydryl group, or an alkylamino or dialkylamino group,
or a salt of said pentapeptide.

2. The composition of claim 1, wherein Pro¹ is 3,4-dehydro Pro.

3. The composition of claim 1, wherein AA¹ is Phe.

4. The composition of claim 3, wherein the pentapeptide is 4-F-Phe-cis- or trans-4-OH-Pro-Ile-Gly-Trp-NH₂ (SEQ ID NO: 41).

5. The composition of claim 3, wherein the pentapeptide is 4-F-Phe-cis- or trans-4-OH-Pro-Leu-Gly-Trp-NH₂ (SEQ ID NO: 42).

6. The composition of claim 3, wherein the pentapeptide is 4-F-Phe-cis- or trans-4-OH-Pro-Arg-Gly-Trp-NH₂ (SEQ ID NO: 43).

7. The composition of claim 1, wherein the composition further comprises at least one skin conditioning agent.

8. The composition of claim 7, wherein the at least one skin conditioning agent comprises at least one moisturizing agent.

9. The composition of claim 1, further comprising at least one topical anti-inflammatory agent.

10. The composition of claim 6, wherein the composition further comprises at least one skin conditioning agent.

11. The composition of claim 10, wherein the at least one skin conditioning agent comprises at least one moisturizing agent.

12. The composition of claim 6, further comprising at least one topical anti-inflammatory agent.

13. A method for the cosmetic treatment of skin, comprising the step of:
applying a topical dermatological composition according to claim 1 to a region of skin.

14. A method for the cosmetic treatment of skin, comprising the step of:
applying a topical dermatological composition according to claim 3 to a region of skin.

15. A method for the cosmetic treatment of skin, comprising the step of:
applying a topical dermatological composition according to claim 6 to a region of skin.

16. A method for manufacturing a topical dermatological composition, comprising the step of:
admixing a dermatologically acceptable vehicle and a pentapeptide of formula (7):
R¹-AA¹-R²-Pro¹-AA²-Gly-AA³-R (7)
wherein Pro¹ is Pro or dehydro-Pro and the N-terminus heterocyclic nitrogen ring of Pro¹ is optionally modified with a substituent selected from the group consisting of cis-4-OH—, trans-4-OH—, cis-3-OH— and trans-3-OH; AA¹ is Phe or Tyr; AA² is selected from the group consisting of Leu, Ile, Arg, D-Arg and Trp; AA³ is Trp; R is a group by which the carboxy-terminus of amino acid AA³ remains unmodified or is modified into a hydroxyalkyl group, a carbamyl group, an alkylcarbamyl group, or an alkoxycarbonyl group; and, R¹ and R² each independently represent a hydrogen atom, a lower alkyl group, a halogen atom, a hydroxyl group, a sulphydryl group, or an alkylamino or dialkylamino group,
or a salt of said pentapeptide.

17. The composition of claim 16, wherein Pro¹ is 3,4-dehydro Pro.

18. The composition of claim 16, wherein AA¹ is Phe.

19. The composition of claim 18, wherein the pentapeptide is 4-F-Phe-cis- or trans-4-OH-Pro-Ile-Gly-Trp-NH₂ (SEQ ID NO: 41).

20. The composition of claim 18, wherein the pentapeptide is 4-F-Phe-cis- or trans-4-OH-Pro-Leu-Gly-Trp-NH₂ (SEQ ID NO: 42).

21. The composition of claim 18, wherein the pentapeptide is 4-F-Phe-cis- or trans-4-OH-Pro-Arg-Gly-Trp-NH₂ (SEQ ID NO: 43).

22. The method of claim 16, further comprising admixing at least one skin conditioning agent.

23. The method of claim 21, further comprising admixing at least one skin conditioning agent.
